(19) United States
(12) Patent Application Publication
(10) Pub. No.: US 2013/0317096 A1
(43) Pub. Date: Nov. 28, 2013

(54) SYNERGISTIC INTERACTION OF AT LEAST ONE VITAMIN E COMPONENT AND TYROSINASE INHIBITORS FOR DERMATOLOGICAL APPLICATIONS

(71) Applicant: Chang Hua Xu, Singapore (SG)

(72) Inventors: Yee Leng Daniel Yap, Singapore (SG); Chang Hua Xu, Singapore (SG)

(73) Assignee: Davos Life Science Pte Ltd, Singapore (SG)

(21) Appl. No.: 13/641,667

(22) PCT Filed: Mar. 22, 2011
(86) PCT No.: PCT/SG2011/000111

§ 371 Date: Jun. 13, 2013

Related U.S. Application Data
(60) Provisional application No. 61/325,011, filed on Apr. 16, 2010.

(51) Int. Cl. A61K 31/355; A61K 31/366; A61K 31/203; A61K 31/19

(52) U.S. Cl. CPC A61K 31/355; A61K 31/19; A61K 31/366; A61K 31/203
USPC 514/458

(57) ABSTRACT

The present invention is directed to a method of treating a dermatological condition or preventing a dermatological condition from occurring or for altering the pigmentation of the skin by administering to a patient a pharmaceutically effective amount of a composition comprising at least one Vitamin E component and at least one additional component different from the Vitamin E component that has anti-tyrosinase activity and/or anti-melanogenesis activity. The present invention is further directed to a pharmaceutical composition comprising at least one Vitamin E component and at least one additional component different from the Vitamin E component that has anti-tyrosinase activity and/or anti-melanogenesis activity.

### CROSS-REFERENCE TO RELATED APPLICATION

This application claims the benefit of priority of U.S. provisional application No. 61/325,011 filed Apr. 16, 2010, the contents of it being hereby incorporated by reference in its entirety for all purposes.

### FIELD OF THE INVENTION

The present invention relates to the field of molecular biology and biochemistry, in particular the field of biochemistry and molecular biology relating to dermatological conditions.

### BACKGROUND OF THE INVENTION

Cutaneous pigmentation is an important protection mechanism against harmful ultraviolet radiation. In the case of an illness or injury for example, the person's skin can change in colour resulting in darker skin tone or colour (hyperpigmentation). Most forms of hyperpigmentation are caused by an excess production of melanin in the body, the substance responsible for colour (pigment). In the body, the formation of pigment melanin occurs within the melanosome of skin melanocytes (Mason, H. S., 1949, J Biol Chem, 181, 803-12; Fitzpatrick, T. B. et. al., 1950, Science, 112, 223-5). This process is regulated by melanogenic enzymes such as tyrosinase and tyrosinase-related protein 1/2 (TRP1/2) (Chen, & Chavin, W. 1966, Nature, 210, 35-7). Specifically, these proteins catalyze the rate limiting, two-part reaction in melanin biosynthesis: the hydroxylation of L-tyrosine to 3,4-dihydroxyphenylalanine (DOPA) and its subsequent oxidation to dopaquinone (Korner, A. & Pawelek, J., 1982, Science, 217, 1163-5). Modulation of tyrosinase activity therefore represents a key process for the regulation of cutaneous pigmentation (Korner and Pawelek, 1982). In addition, because cutaneous pigmentation (melanogenesis process) is a hallmark of melanoma disease, the control of tyrosinase activity may provide a basis for treating patients with this type of cancer.

Dermatological conditions such as those related hyperpigmentation are difficult to treat particularly in dark-skinned individuals or other skin related disorders. Although there are many effective therapeutic modalities available, there are potentially significant side-effects associated with currently available drugs. A common drug for treating a dermatological condition includes hydroquinone, a hydroxyphenolic chemical, which inhibits the enzyme tyrosinase, thereby reducing the conversion of DOPA to melanin. In this regard, some of the other possible mechanisms of action can include the destruction of melanocytes, degradation of melanosomes, and the inhibition of the synthesis of DNA and RNA. There are other phenolic agents, such as Nacetyl-4-cystaminylphenol (NCAP) that are currently being studied and developed. The nonphenolic agents, which include tretinoin, adapalene, topical corticosteroids, azelaic acid, arbutin, kojic acid, and licorice extract, are also used for treating dermatological disorders.

Most of the known methods of treating dermatological conditions have severe side effects on the patient. Therefore, it is an object of the present invention to explore further ways of treating dermatological conditions.

### SUMMARY OF THE INVENTION

In one aspect, the invention provides a method of treating a dermatological condition or preventing a dermatological condition from occurring or for altering the pigmentation of the skin. The method includes administering to a patient a pharmaceutically effective amount of a composition comprising at least one Vitamin E component and at least one additional component different from the Vitamin E component that has anti-tyrosinase activity and/or anti-melanogenesis activity.

In another aspect, the invention provides a pharmaceutical composition comprising at least one Vitamin E component and at least one additional component different from the Vitamin E component that has anti-tyrosinase activity and/or anti-melanogenesis activity. The at least one additional component different from the Vitamin E component is selected from the group consisting of an antioxidant, a tyrosinase inhibitor, a polyphenol, a vitamin, an anti-tyrosinase RNA interference agent, an anti-tyrosinase peptide, or a mixture thereof.

In still another aspect, the invention provides an ointment or cream comprising a pharmaceutical composition as defined above.

In a further aspect, the invention provides a product comprising or consisting of acylated 3,4-dihydro-2,5,7,8-tetramethyl-2-(4,8,12-trimethyl-11-tridecenyl)-2H-1-benzopyran-6-ol.

### BRIEF DESCRIPTION OF THE DRAWINGS

The invention will be better understood with reference to the detailed description when considered in conjunction with the non-limiting examples and the accompanying drawings, in which:

FIG. 1 illustrates the experimental results showing that treatment of B16 melanoma cells with β-tocotrienol (Beta T3), γ-tocotrienol (Gamma T3), δ-tocotrienol (Delta T3), kojic acid and alpha arbutin respectively inhibit B16 melanoma cell viability at high concentrations. The anti-proliferation effect of A) α-tocotrienol (Alpha T3) and β-tocotrienol (Beta T3) respectively, B) γ-tocotrienol (Gamma T3) and δ-tocotrienol (Delta T3) respectively, C) kojic acid and sodium lactate respectively and D) alpha arbutin in B16 melanoma cells was determined by the MTT cell viability assay following 24 hrs of treatment.

FIG. 2 illustrates the Western Blot result of apoptotic molecules in B16 melanoma cells treated with A) δ-tocotrienol (δT3) and γ-tocotrienol (γT3) respectively, B) β-tocotrienol (βT3), kojic acid and alpha arbutin respectively. Note that treatment of B16 melanoma cells with the respective β-tocotrienol, γ-tocotrienol, δ-tocotrienol, kojic acid and alpha-arbutin induced critical apoptotic molecules in a dose-dependent manner (cleaved caspase 3 and PARP).

FIG. 3 illustrates the experimental results showing the activity of tyrosinase in B16 melanoma cells treated with α-tocopherol (αTP), α-tocotrienol (αT3), β-tocotrienol (βT3), δ-tocotrienol (δT3), γ-tocotrienol (γT3), sodium lactate and kojic acid respectively. A) No change of tyrosinase mRNA was observed following treatment of B16 melanoma cells with δ-tocotrienol and γ-tocotrienol, sodium lactate, kojic acid, α-tocopherol (αTP) respectively. B) Western Blot result of tyrosinase protein expression in B16 melanoma cells treated with α-tocopherol (αTP), α-tocotrienol (αT3), β-tocotrienol (βT3), γ-tocotrienol (γT3) and δ-tocotrienol (δT3) respectively. Treatment of B16 melanoma cells with 20 μM of γT3 and δT3 suppresses tyrosinase protein expression. Note that δT3 is the most potent inhibitor of tyrosinase protein.

FIG. 4 illustrates the Western Blot results showing the activity of tyrosinase in B16 melanoma cells treated with the respective palm tocotrienol rich fraction (palm TRF), palm TRF acetate, kojic acid and sodium lactate, γ-tocotrienol (γT3), δ-tocotrienol (δT3), γ-tocotrienol acetate (γT3 acetate) and δ-tocotrienol acetate (δT3 acetate). A) Treatment of B16 cells with 20 μM of Palm TRF and Palm TRF acetate respectively suppresses tyrosinase protein expression. B) Treatment of B16 melanoma cells with 20 μM of kojic acid and sodium lactate respectively does not suppress tyrosinase protein expression. Suppression of tyrosinase is observed only at much higher concentration treatment of kojic acid and sodium lactate respectively. C) Treatment of B16 melanoma cells with 20 μM of γT3 acetate and δT3 acetate respectively suppresses tyrosinase protein expression.

FIG. 5 illustrates the Western Blot results showing the activity of tyrosine in B16 melanoma cells treated with various concentrations of kojic acid, sodium lactate, alpha-arbutin and γ-tocotrienol succinate (γT3 succinate). A) Treatment of B16 cells with up to 100 μM of kojic acid and sodium lactate respectively does not suppress tyrosinase protein expression. B) Treatment of B16 cells with up to 60 μM of arbutin and γT3 succinate respectively does not suppress tyrosinase protein expression. C) Suppression of tyrosinase by alpha arbutin is observed only at much higher concentration treatment (1 mM).

FIG. 6 illustrates the Western Blot result showing the activity of tyrosine in B16 melanoma cells treated with δ-tocotrienol (δT3), γ-tocotrienol (γT3), sodium lactate, kojic acid, α-tocopherol (αTP) and palm tocotrienol rich fraction (palm TRF) respectively beyond 24 hours and 48 hours. Suppression of tyrosinase by γT3 and δT3 in B16 cells is enhanced after a 48 hr incubation period. Conversely, the anti-tyrosinase activities of sodium lactate and kojic acid diminished after 48 hrs. Of note, 20 μM of palm TRF has a lower anti-tyrosinase activity compared to γT3 and δT3, whereas αTP has no impact on the suppression of tyrosinase.

FIG. 7A illustrates the time-dependent tyrosinase activity of B16 melanoma cells measured on days 4 and 15 after treatment with 20 μM of palm tocotrienol rich fraction (palm TRF), 20 μM of γ-tocotrienol (gamma-T3) and 20 μM of δ-tocotrienol (delta-T3) respectively. Bar chart, average for 3 assay measurements; bars, standard deviation.

FIG. 7B illustrates the time-dependent tyrosinase activity of B16 melanoma cells measured on days 5 and 9 after treatment with 20 μM of γ-tocotrienol (gamma T3), 20 μM of δ-tocotrienol (delta-T3), 20 μM of α-tocopherol (alpha TP), 3.5 mM of kojic acid, and 4.5 mM of sodium lactate respectively. Note that gamma T3 significantly suppressed the activity of tyrosinase on day 9.

FIG. 7C illustrates the time-dependent suppression of melanin synthesis in B16 melanoma cells measured on days 5 and 9 after treatment with 20 μM of γ-tocotrienol (gamma T3), 20 μM of δ-tocotrienol (delta T3), 20 μM of α-tocopherol (alpha TP), 3.5 mM of kojic acid, and 4.5 mM of sodium lactate respectively. Note that the gamma T3 significantly suppressed the melanin content up to day 9.

FIG. 8A illustrates the tyrosinase activity of B16 melanoma cells measured on days 5 after treatment with 20 μM of γ-tocotrienol (gamma T3), 20 μM of δ-tocotrienol (delta-T3), 20 μM of tocotrienol rich fraction 92% (T92), 3.5 mM of kojic acid, 4.5 mM of sodium lactate and 1 mM arbutin respectively.

FIG. 8B illustrates the melanin content of B16 melanoma cells measured on day 5 after treatment with 20 μM of γ-tocotrienol (gamma T3), 20 μM of δ-tocotrienol (delta-T3), 20 μM of tocotrienol rich fraction 92% (T92), 3.5 mM of kojic acid, 4.5 mM of sodium lactate and 1 mM arbutin respectively.

FIG. 9 illustrates the anti-pigmentation effect in B16 melanoma cells after treatment with γ-tocotrienol (gamma T3), δ-tocotrienol (delta-T3), α-tocopherol (α-TP), tocotrienol rich fraction 92% (T92), kojic acid and sodium lactate respectively. B16 cells were sub-cultured, treated for gamma- and delta T3, alpha TP, Tocotrienol rich fraction 92%, kojic acid, and sodium lactate then harvested. Photographs of the cell pellets were taken. Note that treatments of B16 cells with 20 μM of gamma- and delta-T3, T92, and 3.5 mM kojic acid led to lighter cell pigmentation. Conversely, 20 μM of alpha TP, kojic acid, and sodium lactate produced cell pellets with comparable pigmentation level to controls.

FIG. 10 illustrates the anti-pigmentation effect in B16 melanoma cells after treatment with α-tocopherol (α-TP), γ-tocotrienol (γT3), δ-tocotrienol (δT3), tocotrienol rich fraction 92% (T92), and arbutin respectively. B16 cells were sub-cultured, treated for gamma- and delta T3, alpha TP, Tocotrienol rich fraction 92% (T92), alpha arbutin then harvested. Photographs of the cell pellets were taken. Note that treatments of B16 cells with 20 μM of gamma- and delta-T3, T92, and 1 mM alpha arbutin led to lighter cell pigmentation. Conversely, 20 μM of alpha TP, alpha arbutin produced cell pellets with comparable pigmentation level to controls.

FIG. 11 illustrates the anti-pigmentation effect and tumor shrinkage of nude mice in which pre-treated B16 melanoma cells were xenografted onto the flank of the nude mice. 5×10⁵ B16 cells pre-treated with 20 μM of γ-tocotrienol (γT3) for 1 week were xenografted on the flank of nude mice. This was followed by a 2-week supplementation of γT3 at the dose of 50 mg/kg/day. Photographs of the solid tumors were taken at the end of a 2-week treatment.

FIG. 12 illustrates the anti-pigmentation effect and tumor shrinkage of nude mice in which pre-treated B16 melanoma cells were xenografted onto the flank of the nude mice. 5×10⁵ B16 cells pre-treated with 20 μM of δ-tocotrienol (δT3) and palm tocotrienol rich fraction 92% (T92) respectively for 1 week were xenografted on the flank of nude mice. This was followed by a 2-week supplementation of the respective δT3 and T92 at the dose of 50 mg/kg/day. Photographs of the solid tumors were taken at the end of a 2-week treatment.

FIG. 13 illustrates the tumor size of the nude mice treated with γ-tocotrienol (gammaT3), δ-tocotrienol (gamma T3) and palm tocotrienol rich fraction (Palm TRF) respectively. The tumor size was measured at the start and end of the experiments.

FIG. 14 illustrates the de-pigmentation property of palm tocotrienol rich fraction (TRF) tested in human trials. Photographs of aged spots on the face of one subject, before and after 1 month treatment using Kose Prime cream containing 2% palm TRF. The control subjects did not show improvement in age spot (n=13).

FIG. 15A illustrates the effect of UV on tyrosinase activity of B16 melanoma cells pre-treated with palm Tocotrienol rich fraction 92% (T92), γ-tocotrienol (γT3), δ-tocotrienol (δT3) and T92 acetate (T92Ac), followed by 10 minutes of UV exposure (short and long wave UV). Bar chart, average for 3 assay measurements; bars, standard deviation.

FIG. 15B illustrates the effect of UV on the melanin content of B16 cells pre-treated with palm tocotrienol rich fraction 92% (T92), γ-tocotrienol (γT3), δ-tocotrienol (δT3) and T92 acetate (T92Ac), followed by 10 minutes UV exposure (short and long wave UV). Bar chart, average for 3 assay measurements; bars, standard deviation. Note that the γT3, δT3 and T92 significantly suppressed the melanin content. Bar chart, average for 3 assay measurements; bars, standard deviation.

FIG. 16A illustrates the experimental result showing the viability of B16 melanoma cells at different time periods. UV exposure beyond 60 minutes induced apoptosis in B16 cells.

FIG. 16B illustrates the Western Blot result of apoptotic molecules in B16 melanoma cells after being exposed to UV for 10 minutes, 60 minutes and 12 hours. Note that B16 melanoma cells exposed to UV for 12 hours induced critical apoptotic molecules (cleaved caspase 3 and PARP).

FIGS. 16C to E illustrate effect of tyrosinase protein expression after B16 cells treated with γ-tocotrienol (γT3), δ-tocotrienol (δT3), palm tocotrienol rich fraction 92% (palm TRF) and palm tocotrienol rich fraction acetate (palm TRF acetate) were exposed to UV at 1 minute, 10 minutes, 30 minutes and 60 minutes. γT3, δT3 and palm TRF significantly suppressed the tyrosinase protein expression of B16 cells exposed to UV.

FIG. 17A illustrates the synergistic effect in tyrosinase activity and melanin content of B16 melanoma cells treated with palm tocotrienol rich fraction 92% (palm TRF) and kojic acid, compared to either agent alone. B16 cells were treated with 20 μM of palm Tocotrienol rich fraction 92% (palm TRF) and 0.05% of either sodium lactate (4.5 mM) or kojic acid (3.5 mM) for 24 hrs. The suppression of tyrosinase activity and melanin content following co-treatment were significantly greater than the cells treated with either agent alone.

FIG. 17B illustrates the Western blot result showing the synergistic effect of tyrosinase protein expression in B16 melanoma cells when treated with γ-tocotrienol (γT3) and kojic acid or sodium lactate, compared to either agent alone. Based on this result, 5 μM of γT3 co-treatment with 1 mM of either sodium lactate or kojic acid resulted in enhanced suppression of tyrosinase protein.

FIG. 17C illustrates the Western blot result showing the synergistic effect of tyrosinase protein expression in B16 melanoma cells when co-treated with δ-tocotrienol (δT3) and kojic acid or sodium lactate, compared to either agent alone. Based on this result, 5 μM of δT3 co-treatment with 1 mM of either sodium lactate or kojic acid resulted in enhanced suppression of tyrosinase protein.

FIGS. 18A to C illustrate the Western blot result showing the synergistic effect of tyrosinase protein expression in B16 melanoma cells when co-treated with γ-tocotrienol (γT3) (10 μM) and alpha arbutin (50 μM) (FIG. 18A), or hydroquinone (20 μM) (FIG. 18B), or L-gluthathione (10 μM) (FIG. 18C), compared to either agents alone.

FIG. 18D illustrates the Western blot result showing the tyrosinase protein expression in B16 melanoma cells transfected with mi434-5P microRNA and treated with γ-tocotrienol (γT3), compared with non-transfected B16 cells.

FIG. 18E illustrates the Western blot result showing the tyrosinase expression in B16 melanoma cells when co-treated with γ-tocotrienol (γT3) (10 μM) and retinoic acid (2 nM) compared with either component alone.

### DETAILED DESCRIPTION OF THE INVENTION

In a first aspect the present invention refers a method of treating a dermatological condition or preventing a dermatological condition from occurring or for altering the pigmentation of the skin by administering to a patient a pharmaceutically effective amount of a composition comprising at least one Vitamin E component and at least one additional component different from the Vitamin E component that has anti-tyrosinase activity and/or anti-melanogenesis activity.

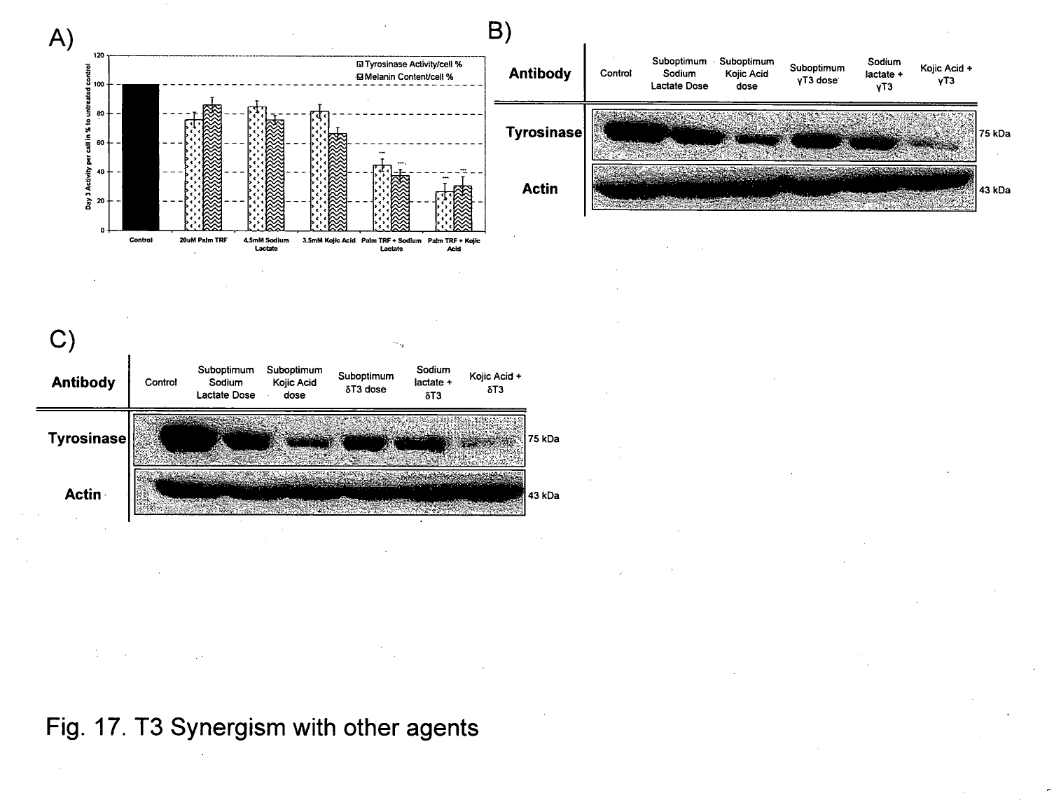

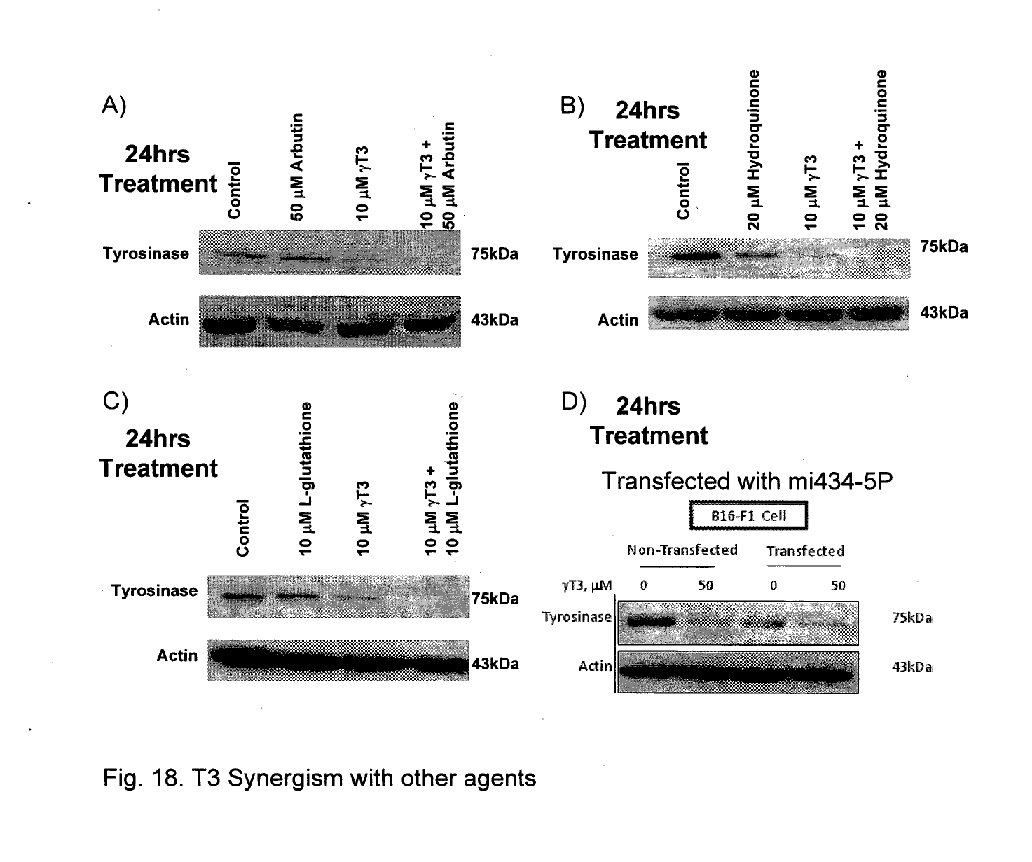

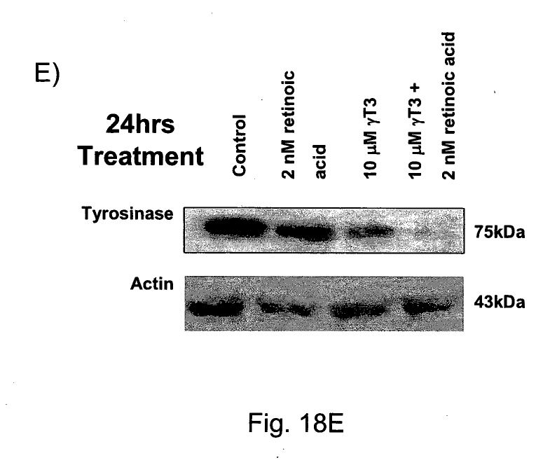

It has been demonstrated that a composition comprising at least one Vitamin component such as γ-tocotrienol (γT3), δ-tocotrienol (δT3) or tocotrienol rich fraction (TRF) for example, suppress constitutive melanin synthesis in cells such as B16 melanoma cells, as a result of suppressing the constitutive activation of tyrosinase protein. It has also been demonstrated that a Vitamin E component referred to in the present invention, such as γ-tocotrienol (γT3) or δ-tocotrienol (δT3), possess synergistic interaction with an additional component different from the Vitamin E component having anti-tyrosinase activity and/or anti-melanogenesis activity for example but are not limited to, sodium lactate, kojic acid or retinoic acid. These findings are supported by in vitro as well as in vivo data as can be observed from the experimental results referred to herein. The general principal of this aspect of the present invention can also be illustrated in FIGS. 17 and 18 based on examples in which a composition comprising at least one Vitamin E component for example, γ-tocotrienol (γT3), δ-tocotrienol (δT3) with another component different from the Vitamin E component that has anti-tyrosinase activity and/or anti-melanogenesis activity such as sodium lactate, kojic acid, alpha arbutin, hydroquinone, L-gluthathione, or a mi434-5P microRNA molecule, demonstrated significant tyrosinase protein suppression compared to using either component alone.

The term “treat” or “treating” as used herein is intended to refer to providing a pharmaceutically effective amount of a composition comprising at least one Vitamin E component and at least one additional component different from the Vitamin E component that has anti-tyrosinase activity and/or anti-melanogenesis activity, sufficient to act prophylactically to prevent the development of a weakened and/or unhealthy state; and/or providing a subject or patient with a sufficient amount of the composition or medicament thereof so as to alleviate or eliminate a disease state/disorder and/or the symptoms of a disease state/disorder, and a weakened and/or unhealthy state.

With “preventing a dermatological condition from occurring” it is referred to the act of preventing or hindering a dermatological condition from occurring. In the present case, administering a composition referred to herein has the effect that the dermatological condition cannot develop in a patient or an animal body. Prevention is to be differentiated from “treatment” in which a composition referred to herein would be used for treating a dermatological condition which already exist in the patient or animal body or in other words for the treatment of a patient or animal body already suffering from a dermatological condition.

In general, a “dermatological condition” is considered to refer to any condition, disorder or disease, such as cancer, cosmetic and ageing conditions associated with the skin, far, hair, nails, oral and genital membranes and glands. A dermatological disorder can manifest in the form of visible lesions, pre-emergent lesions, pain, sensitivity to touch, irritation, inflammation, or the like. Dermatological disorders include but are not limited to disorders of the cutaneous and pilosebaceous unit or the process of keratogenesis. For example, a dermatological disorder can be a disorder of the epidermis or dermis, or within and surrounding a pilosebaceous unit, which is located within the epidermis, dermis, subcutaneous layer, or a combination thereof. Examples of dermatological disorders include, but are not limited to, acne, alopecia, psoriasis, seborrhea, ingrown hairs and pseudofolliculitis barbae, hyperpigmented skin, cutaneous infections, lichen planus, Graham Little Syndrome, periorificial dermatitis, rosacea, hidradenitis suppurativa, dissecting cellulitis, systemic lupus erythematosus, discoid lupus erythematosus, and the like.

In some embodiments, the types of dermatological disorder which can be treated or prevented using the composition referred to herein can for example include a skin disorder. The skin disorder can, for example, include darkening of skin caused by increased melanin, skin hyperpigmentation, skin inflammation, skin acne vulgaris, wound healing, skin photoaging, skin wrinkles, smoker's melanosis, melasma, acanthosis nigricans, Cushing's disease, Addison's disease, linea nigra, mercury poisoning, to name only a few.

With “altering the pigmentation of the skin”, it is referred to a change in the natural colour (pigment) of the skin. It can be referred to a decrease in the level or amount of pigmentation of the skin. In some embodiments, administering a composition referred to herein can reduce an actual skin impairment of dark colour, or can for example prevent or stop a dark area of the skin from enlarging. An actual skin impairment can for example be caused by age, excessive sun exposure, or a disease or disorder leading to dark skin areas. These diseases can for example include any of the dermatological disorders mentioned herein. In other embodiments, administering a composition referred to herein can reduce a perceived skin impairment of dark colour. This means that the skin impairment can be perceived by an individual such that his or her skin shade is dark, who does not necessarily have an actual skin impairment but a (cosmetic) desire to lighten the skin shade.

As described herein, for the treatment of a dermatological condition or prevention of a dermatological condition from occurring or alteration of the pigmentation of the skin, a composition comprising at least one Vitamin E component and at least one additional component different from the Vitamin E component that has anti-tyrosinase activity and/or anti-melanogenesis activity is used. Vitamin E is composed of two main components—Tocopherols (T) and Tocotrienols (T3). Tocotrienols (T3) are found mainly in palm oil. Together with tocopherols (T), they provide a significant source of anti-oxidant activity to all living cells. This common anti-oxidant attribute reflects the similarity in chemical structures of the tocotrienols and the tocopherols, which differ only in their structural side chain (contains farnesyl for tocotrienol or saturated phytyl side chain for tocopherol). The common hydrogen atom from the hydroxyl group on the chromanol ring acts to scavenge the chain-propagating peroxyl free radicals. Depending on the locations of methyl groups on their chromanol ring, tocopherols and tocotrienols can be distinguished into four isomeric forms: alpha (α), beta (β), gamma (γ), and delta (δ).

Different tocopherol and tocotrienol isoforms exist (see Formula I and II). Tocopherols consist of a chromanol ring and a 15-carbon tail derived from homogentisate (HGA) and phytyl diphosphate, respectively. On the other hand, tocotrienols differ structurally from tocopherols by the presence of three trans double bonds in the hydrocarbon tail. Formula I and Formula II and the description following it provide an overview about the known isoforms of tocopherols (T) and tocotrienols (T3).

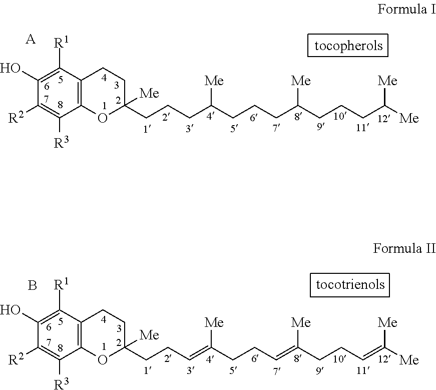

Formula I (A): R¹=R²=R³=Me (CH₃), known as α(alpha)-tocopherol, is designated α-tocopherol or 5,7,8-trimethyltocol; R¹=R³=Me; R²=H, known as, β(beta)-tocopherol, is designated, β-tocopherol or 5,8-dimethyltocol; R¹=H; R²=R³=Me, known as γ(gamma)-tocopherol, is designated γ-tocopherol or 7,8-dimethyltocol; R¹=R²=H; R³=Me, known as δ(delta)-tocopherol, is designated δ-tocopherol or 8-methyltocol. Formula II (B): R¹=R²=R³=H, 2-methyl-2-(4,8,12-trimethyltrideca-3,7,11-trienyl)chroman-6-ol, is designated tocotrienol; R¹=R²=R³=Me, formerly known as (1 or (2-tocopherol, is designated 5,7,8-trimethyltocotrienol or α(alpha)-tocotrienol. The name tocochromanol-3 has also been used; R¹=R³=Me; R2=H, formerly known as e-tocopherol, is designated 5,8-dimethyltocotrienol or β(beta)-tocotrienol; R¹=H; R²=R³=Me, formerly known as γ-tocopherol, is designated 7,8-dimethyltocotrienol or (gamma)γ-tocotrienol. The name plastochromanol-3 has also been used; R¹=R²=H; R³=Me is designated 8-methyltocotrienol or δ(delta)-tocotrienol.

Another Vitamin E component is α-tocomonoenol which exists in different isomeric forms and δ-tocomonoenol. One isomeric form of α-tocomonoenol, namely 3,4-dihydro-2,5,7,8-tetramethyl-2-(4,8,12-trimethyl-11-tridecenyl)-2H-1-benzopyran-6-ol can be isolated, e.g., from palm oil tree, such as Elaeis guineensis jacq. Another isomeric form of α-tocomonoenol, namely 3,4-dihydro-2,5,7,8-tetramethyl-2-(4,8,12-trimethyl-12-tridecenyl)-2H-1-benzopyran-6-ol can be isolated from, e.g., pacific salmon (Oncorhynchus keta) (Yamamoto Y. et al., J. Nat. Prod. 1999, 62, 1685-1687). δ-tocomonoenol can be isolated from Actinidia chinensis (kiwi) fruits (Fiorentino A et. al, Food Chemistry, 2009, 115, 187-192). The structure of δ-tocomonoenol has also been elucidated as 2,8-dimethyl-2-(4,8,12-trimethyltridec-11-enyl)chroman-6-ol.

In some embodiments, a Vitamin E component referred to herein can be an acylated Vitamin E component. The acylated Vitamin E component can be an acetylated Vitamin E component. Acylation generally refers to a process of adding an acyl group to a compound. In some embodiments, the acylation reaction can be carried out using an acylating agent, such as an acid anhydride or acyl halide. For example, acylation of a Vitamin E component leads to esterification of a phenolic hydroxyl group comprised in a tocopherol, tocotrienol or tocomonoenol for example, to result in a tocopherol acylate, tocotrienyl acylate or tocomonoenol acylate. In some embodiments, the acylated tocopherol, tocotrienol or tocomonoenol can be an acetylated tocopherol, tocotrienol or tocomonoenol.

An acyl group in any of the acylating agents referred to herein can be derived from aliphatic carboxylic acids, for example, linear or branched chain alkanoic acids, e.g. as C₁-C₇ alkanoic acids, such as acetic acid, propionic acid, butyric acid and pivalic acid or from higher alkanoic acids (fatty acids) with up to 20 carbon atoms, such as palmitic acid, or from aromatic carboxylic acids, such as benzoic acid. In the context of this embodiment, the carboxylic anhydride can be one selected from acetic anhydride, propionic anhydride, butyric anhydride, maleic anhydride, chloroacetic anhydride, succinic anhydride, phthalic anhydride and citraconic anhydride not to mention a few.

Examples of acyl halides can include, but are not limited to linear or branched chain alkanoyl chlorides, such as acetyl, propionyl and butyryl chloride, and of aromatic halides, such as benzoyl chloride.

Acylation of at least one Vitamin E component referred to herein can be carried out in the presence of a catalyst, for example an acidic or a base catalyst. An acid or base catalyst used in the acylation of the Vitamin E component can refer to any respective Lewis base or acid catalyst so long as the catalyst performs its desired function in the acylation reaction. The basic catalyst can be any one of the following compounds of N, P, As, Sb and Bi in oxidation state 3, compounds of O, S, Se and Te in oxidation state 2, such as ether, ketones or sulphoxides, or carbon monoxide. In this context, the Lewis base catalyst can be one selected from pyridine, triethylamine, dimethylaminopyridine (DMAP), N-methylimidazole, 3-(1-methyl-2-pyrrolidinyl) pyridine, or 4-pyrrolidinopyridine (PPY) not to mention a few. The acid catalyst can include but is not limted to NH₃, B₂H₆, BF₃, Al₂Cl₆, AlF₃, SiF₄, PCl₅, SF₄, metal ions forming solvates, such as [Mg(H₂O)₆]²⁺ or [Al(H₂O)₆]³⁺, (1-H-3-methyl-imidazolium bisulfate), 1-hexyl-3-methyl-imidazolium bisulfate ([hmim][HSO₄]), 1-butyl-3-methylimidazolium dihydrogen phosphate ([bmim][H₂PO₄]), 1-[2-(2-hydroxy-ethoxy)ethyl]-3-methyl-imidazolium bisulfate ([heemim][HSO₄]), 1-butyl-3-methyl-imidazolium chloroaluminate ([bmim]Cl 2AlCl₃) and 1-butyl-3-methyl-imidazolium bisulphate ([bmim][HSO₄]).

In one embodiment, the acylating agent can be used in excess (such as 3 to 10 fold molar ratio to the vitamin E component) in the acylating reaction in comparison to the tocotrienol and/or tocopherol.

In one embodiment, acylation of at least one Vitamin E component referred herein can include carrying out the catalyzed acylation reaction of tocotrienol for example, at a pressure above atmospheric pressure. In this context, the pressure can be in the range of at least 2 bar or at least 5 bar, or between about 2 bar to about 12 bar, or between about 5 bar to 10 bar. In some embodiments, the acylation reaction can be carried out under an inert atmosphere. In the context of this embodiment, the inert atmosphere can be a nitrogen or halogenide, such as argon.

In one embodiment, acylation of the at least one Vitamin E component can include carrying out the catalyzed acylation reaction of the Vitamin E component for example tocotrienol at ambient temperature. In general ambient temperature is understood to be a temperature in a range of between about 15° C. to about 35° C. In one embodiment, ambient temperature is a temperature between about 20° C. to about 30° C. or 25° C. to about 30° C.

In some embodiments, the acylation reaction of a Vitamin E component referred to herein can be carried out for a time between about 10 minutes to about 60 minutes, or between 10 minutes to about 180 minutes. In other embodiments, the acylation reaction can be carried out between about 15 minutes to about 30 minutes.

Other methods for acylating a Vitamin E component as referred to herein are within the knowledge of a person of average skill in the art and can also be described in U.S. Pat. No. 7,169,973, U.S. Pat. No. 6,239,294, U.S. Pat. No. 7,135,580, and U.S. Pat. No. 5,523,420. As a non-limiting example, a Vitamin E component such as tocotrienol-rich fraction (TRF) can be mixed with a carboxylic anhydride for example acetic anhydride and stirred under N₂ at room temperature for a predetermined period of time for example about 2 hrs, in the presence of a catalyst such as pyridine (see also O'Byrne, D., et al, Free Radical Biology & Medicine, 2002, 29, 834-45). The extra anhydride and its corresponding acid and the catalyst can be removed by distillation procedures known in the art including vacuum distillation for example. The residual TRF acetate can be further purified by separation methods known in the art, including for example, column chromatography, distillation, not to mention a few.

The at least one Vitamin E component used in the composition referred to herein can comprise or consist of at least one of tocopherol, tocotrienol, tocomonoenol, acylated tocopherol, acylated tocotrienol and acylated tocomonoenol. The Vitamin E component can also comprise or consist of a mixture of tocopherol, tocotrienol, tocomonoenol, acylated tocopherol, acylated tocotrienol and acylated tocomonoenol. In some embodiments, the at least one Vitamin E component used herein can be a mixture of at least one tocopherol and at least one tocotrienol or a mixture of at least one acylated tocopherol and at least one acylated tocotrienol. The at least one Vitamin E component can be a tocotrienol-rich fraction (TRF) or an acylated TRF, for example TRF acetate. A tocotrienol-rich fraction typically refers to a mixture of different isomers of tocotrienol and tocopherols, for example, α-tocopherol, α-tocotrienol, β-tocotrienol, γ-tocotrienol, and δ-tocotrienol. The tocotrienol-rich fraction can further include other components such as plant phytosterols, carotenoids and squalene to name only a few. Tocotrienol-rich fraction can for example be obtained from palm oil, rice bran, or grape seed.

In some embodiments, the at least one Vitamin E component used in the composition referred to herein can comprise γ-tocotrienol, δ-tocotrienol, tocotrienol-rich fraction (TRF), acylated γ-tocotrienol, acylated δ-tocotrienol, acylated TRF or mixtures thereof.

The additional component different from the Vitamin E component can refer to any component as long as the component possesses anti-tyrosinase activity and/or anti-melanogenesis activity. This additional component can for example be an antioxidant, a tyrosinase inhibitor, a polyphenol, a vitamin, an anti-tyrosinase RNA interference agent, an anti-tyrosinase peptide, or a mixture of any of the components, to mention only a few.

With “anti-melanogenesis activity”, it is generally referred to a component that has the capability to prevent or inhibit the synthesis of melanin in a cell. The additional component can for example be an agent that modulates or inhibits or interferes with melanogenesis. In this context, the additional component different from the Vitamin E component referred to herein can have anti-melanogenesis activity but does not necessarily have anti-tyrosinase activity. Without wishing to be bound by any theory, such an additional component that has anti-melanogensis activity can, for example, relate to a mechanism other than a mechanism that modulates tyrosinase activity. Such a mechanism can for example include preventing melanin transfer from melanocytes to keratinocytes; or reducing melanin-related metabolites to non-colour forms; to mention only a few. The additional component different from the Vitamin E component can also have both anti-tyrosinase activity and anti-melanogenesis activity.

Anti-tyrosinase peptides may also include pre- or pro-proteins or mature proteins, including polypeptides or proteins that are capable of being directed to the endoplasmic reticulum (ER), a secretory vesicle, a cellular compartment, or an extracellular space typically, e.g., as a result of a signal sequence, however, proteins released into an extracellular space without necessarily having a signal sequence are also encompassed. Generally, the polypeptides undergo processing, e.g., cleavage of a signal sequence, modification, folding, etc., resulting in a mature form. If an anti-tyrosinase peptide is released into the extracellular space, it can undergo extracellular processing to produce a “mature” protein. Release into the extracellular space can occur by many mechanisms, including, e.g., exocytosis, and proteolytic cleavage.

Anti-tyrosinase peptides may also be “altered,” resulting in “variations,” and may contain deletions, insertions, or substitutions of amino acid residues that produce a silent change and result in functionally equivalent proteins. Deliberate amino acid substitutions may be made based on similarity in polarity, charge, solubility, hydrophobicity, hydrophilicity, and/or the amphipathic nature of the residues, as long as the biological or immunological activity of the anti-tyrosinase polypeptide is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

Anti-tyrosinase peptides can be prepared in any manner known in the art. For example, naturally occurring anti-tyrosinase peptides can be isolated, recombinantly produced, synthetically produced, or produced by any combination of these methods. For example, a recombinantly produced version of an anti-tyrosinase peptide, including a secreted polypeptide, can be purified using techniques described herein or otherwise known in the art. See Martin F H, et. al., Primary structure and functional expression of rat and human stem cell factor DNAs. Cell 63:203, 1990. An anti-tyrosinase peptide also may be purified from natural, synthetic or recombinant sources or otherwise known in the art, such as, e.g., using an antibody raised against anti-tyrosinase peptide or a peptide sequence fused to anti-tyrosinase peptide. See, e.g., U.S. Pat. No. 6,759,215 or 6,207,417. As a non-limiting example, the anti-tyrosinase peptide can include gluthathione, L-gluthathione, including their derivatives known to persons skilled in the art.

A tyrosinase inhibitor can be obtained either from natural or synthetic sources. Tyrosinase is a copper-containing enzyme that catalyzes the reaction of melanin synthesis. Without wishing to be bound by theory, a tyrosinase inhibitor mainly acts by interfering in the synthesis of melanin, regardless whether there is any direct inhibitor/enzyme interaction. Besides the common tyrosinase inhibitors such as kojic acid, tyrosinase inhibitors can generally be classified into five major classes, including a) polyphenols, b) benzaldehyde and benzoate derivatives, c) long-chain lipids and steroids, d) other natural or synthetic inhibitors, e) and irreversible inactivators based on either the chemical structures or the inhibitory mechanism.

Polyphenols represent a diverse group of compounds containing multiple phenolic functionalities and are widely distributed in nature. An example of polyphenol includes hydroquinone. Another example of polyphenol include flavonoids which are benzo-γ-pyrone derivatives consisting of phenolic and pyrene rings. Flavonoids can be subdivided into seven major groups, including flavones, flavonols, flavanones, flavanols, isoflavonoids, chalcones, and catechin. Different classes of flavonoids are distinguished by additional oxygen-heterocyclic rings, by positional differences of the B ring, and by hydroxyl, methyl, isoprenoid, and methoxy groups distributed in different patterns about the rings. Without wishing to be bound by theory, the structure of flavonoids is compatible with the roles of both substrates and (presumably competitive) inhibitors of tyrosinase. In addition to flavonoids, other polyphenols, which can be used as tyrosinase inhibitors, contain stilbenes and coumarin derivatives. Derivatives of polyphenol can also be used as tyrosinase inhibitors as referred to herein. Such polyphenol derivatives can include (phenolic) glycosides, for example, hydroquinone glycosides. A non-limiting example of hydroquinone glycoside includes alpha arbutin.

In some embodiments, the at least one additional component different from the Vitamin E component referred to herein can be one selected from the group consisting of vitamin A, vitamin A derivatives, vitamin B, vitamin B derivatives, vitamin C, vitamin C derivatives, quinones, hydroquinone, lactates, kojic acid, kojic acid derivatives, alpha hydroxy acids, arbutin, glycolic acid, hydroquinone, glutathione, L-glutathione, azelaic acid, glucocorticoids, Mulberry extract, mitracarpus scaber extract, Cucumis sativus extract, licorice extract, pomegranate extract, uva ursi (bearberry) extract, hexamethylene bisacetamide, sodium butyrate, dimethyl sulfoxide, synthetic hydroxyl substituted phenyl naphthalenes and derivatives, monophenols, such as 2,6-di-tert-butyl-4-methylphenol, 2,6-di-tert-butyl-4-ethylphenol and the like; and diphenols, such as 4,4′-methylenebis(2,6-di-tert-butylphenol), 2,2′-methylenebis(4-ethyl-6-tert-butylphenol), retinoic acid, tunicamycin, N-acetyl glucosamine, hyaluronic acid, tranexamic, placental extract, ellagic Acid, EDTA, phytic acid, aleosin, thioctic Acid, Protease Activated Receptor (PAR2) and soybean trypsin inhibitor, to mention only a few. In this context, when desired, any lactate or lactate salt can be used as the additional component different from the Vitamin E component, so long as it has anti-tyrosinase activity. Examples of a lactate or lactate salt can include but is not limited to potassium lactate, sodium lactate, magnesium lactate, ammonium lactate and calcium lactate.

In other embodiments, the at least one additional component different from the Vitamin E component referred to herein can for example include one of sodium lactate, hydroquinone, L-glutathione, kojic acid, arbutin, retinoic acid, including derivatives thereof, to mention only a few. In this context, suitable kojic acid derivatives that are known to persons skilled in the art can be used. Examples of such kojic acid derivatives include but are not limited to kojic acid dipalmitate (U.S. Pat. No. 5,824,327), kojic acid esters (U.S. Pat. No. 4,278,656), kojic acid and cyclodextrin (U.S. Pat. No. 4,847,074).

In some embodiments, the additional component different from the Vitamin E component can include a skin whitening agent. The “skin whitening agent” as used herein refers to any compound or substance that can have the effect of altering the pigment of the skin as long as the agent has anti-tyrosinase activity and/or anti-melanogenesis activity. The skin whitening agent can for example be used in the composition as described herein in order to reduce an actual skin impairment of dark colour. When desired, the skin whitening agent can for example, also be used in the composition as described herein in order to reduce an individual's perceived skin impairment of dark colour. This means that the individual does not necessarily have an actual skin impairment but has the desire to lighten the skin shade. Examples of skin whitening agents that can be used in the composition as referred to herein can include but are not limited to any of the exemplary additional components mentioned above.

In some embodiments, the at least one additional component different from the Vitamin E component can comprise an anti-tyrosinase RNA interference agent. An anti-tyrosinase RNA interference agent (for e.g. an interfering ribonucleic acid) can refer to any compound that modulates the activity of a nucleic acid encoding an tyrosinase. In this context, the term “nucleic acid”, “nucleotide”, “nucleotide molecule” or “oligonucleotide” refers to polynucleotides such as deoxyribonucleic acid (DNA), and ribonucleic acid (RNA). The term should also be understood to include, as applicable to the embodiment being described, single-stranded (such as sense or antisense) and double-stranded polynucleotides. Besides ribose or deoxyribose the sugar groups of the nucleotide subunits may be also modified derivatives thereof such as 2′-O-methyl ribose. The nucleotide subunits of an oligonucleotide may be joined by phosphodiester linkages, phosphorothioate linkages, methyl phosphonate linkages or by other rare or non-naturally-occurring linkages that do not prevent hybridization of the oligonucleotide. Furthermore, an oligonucleotide may have uncommon nucleotides or non-nucleotide moieties.

The anti-tyrosinase RNA interference agent can for example, include interfering RNAs, short hairpin RNAs and micro RNAs. These anti-tyrosinase RNA interference agents have become a powerful tool to “knock down” specific genes. RNAi methodology makes use of gene silencing or gene suppression through RNA interference (RNAi), which occurs at the posttranscriptional level and involves mRNA degradation. RNA interference represents a cellular mechanism that protects the genome. siRNA and miRNA molecules mediate the degradation of their complementary RNA by association of the siRNA with a multiple enzyme complex to form what is called the RNA-induced silencing Complex (RISC). The siRNA or miRNA becomes part of RISC and is targeted to the complementary RNA species which is then cleaved. siRNAs are perfectly base paired to the corresponding complementary strand, while miRNA duplexes are imperfectly paired. Activation of RISC leads to the loss of expression of the respective gene. Interfering ribonucleic acids may not exceed about 100 nt in length, and typically does not exceed about 75 nt length. Where the interfering ribonucleic acid is a duplex structure of two distinct ribonucleic acids hybridized to each other, e.g., a siRNA, the length of the duplex structure typically ranges from about 15 to 30 bp, usually from about 15 to 29 bp. Where the RNAi agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. In some embodiments, the miRNA is mi434-5P miRNA (see Wu David T. S. et. al, Clinical, Cosmetic and Investigational Dermatology, 2008, 1, 19-35).

The anti-tyrosinase RNA interference agent can also be an antisense RNA. An antisense RNA as used herein refers to a single stranded RNA sequence which is complementary to a sequence of bases in a messenger RNA (mRNA). By “complementary” is meant that the nucleotide sequences of similar regions of two single-stranded nucleic acids, or to different regions of the same single-stranded nucleic acid have a nucleotide base composition that allow the single strands to hybridize together in a stable double-stranded hydrogen-bonded region. When a contiguous sequence of nucleotides of one single-stranded region is able to form a series of “canonical” hydrogen-bonded base pairs with an analogous sequence of nucleotides of the other single-stranded region, such that A is paired with U or T and C is paired with G, the nucleotides sequences are “perfectly” complementary. When an antisense RNA is introduced in a cell, for example B16 cells used in the present invention, the antisense RNA can inhibit translation of a complementary mRNA which encodes the tyrosinase protein. By complementary base pairing between the antisense RNA and the mRNA sequence, the translation pathway can be obstructed.

With the phrase “nucleic acid hybridization” is meant the process by which two nucleic acid strands having completely or partially complementary nucleotide sequences come together under predetermined reaction conditions to form a stable, double-stranded hybrid with specific hydrogen bonds. Either nucleic acid strand may be a deoxyribonucleic acid (DNA), a ribonucleic acid (RNA), or an analog of one of these nucleic acids; thus hybridization can involve RNA:RNA hybrids, DNA:DNA hybrids, or RNA:DNA hybrids.

Where the mammalian target cells are in vivo, the anti-tyrosinase RNA interference agent that is used in the method of the present invention can be administered to the mammalian host using any convenient protocol which is known to a person skilled in the art. The following discussion provides a review of representative nucleic acid, such as siRNA, administration protocols that may be employed. The nucleic acids may be introduced into tissues or host cells by any number of routes, including viral infection, microinjection, or fusion of vesicles.

Jet injection may also be used for intra-muscular administration, as described by Furth, P. A., Shamay, A., et al. (1992) “Gene transfer into somatic tissues by jet injection” Anal Biochem, vol. 205, p. 365-368. The nucleic acid may be coated onto gold microparticles and delivered intradermally by a particle bombardment device or “gene gun” as described in the literature (see, for example, Tang, D. C., De Vit, M., et al., (1992) “Genetic immunization is a simple method for eliciting an immune response” Nature, vol. 356, p. 152-154), where gold microparticles are coated with the DNA, then bombarded into skin cells. The use of nanoparticles for delivering siRNA is another suitable approach for cell-specific targeting. This method has been described for example by Weissleder, R., Kelly, K., et al. (2005) “Cell-specific targeting of nanoparticles by multivalent attachment of small molecules” Nature Biotech, vol. 23, p. 1418-1423.

One illustrative example of delivering an anti-tyrosinase RNA interference agent for example siRNA into selected cells in vivo is its non-covalent binding to a fusion protein of a heavy-chain antibody fragment (F_{ab}) and the nucleic acid binding protein protamin (Song, E., Zhu, P., et al. (2005) “Antibody mediated in vivo delivery of small interfering RNAs via cell-surface receptors” Nature Biotech, vol. 23, p. 709-717). Another illustrative example of delivering a siRNA molecule into selected cells in vivo is its encapsulation into a liposome. Morrissey, D., Lockridge, J., et al. “Potent and Persistent In Vivo Anti-HBV Activity of Chemically Modified siRNAs” Nature Biotech (2005), vol. 23, p. 1002-1007) for instance used a stable nucleic acid-lipid-particle, coated with a polyethylene glycol-lipid conjugate, to form liposomes for intravenous administration.

Yet a further example of delivering an RNAi agent to a selected malignant target cell is the use of a biological vehicle such as a bacterium or a virus (e.g. adenovirus) that includes the respective nucleic acid molecule. Xiang, S., Fruehauf, J., et al. (2006) “Short hairpin RNA-expressing bacteria elicit RNA interference in mammals” Nature Biotech, vol. 24, p. 697-702, have for instance used this approach by administering the bacterium E. coli, which transcribed from a plasmid inter alia both shRNA and invasin, thus permitting entry into mammalian cells and subsequent gene silencing therein.

Expression vectors may be used to introduce siRNA into the desired cells. In addition, the oligonucleotide can be fed directly to, injected into, the host organism containing the target gene, tyrosinase coding gene. The siRNA may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution. The agent may be introduced in an amount which allows delivery of at least one copy per cell. Higher doses (e.g., at least 5, 10, 100, 500 or 1000 copies per cell) of the agent may yield more effective inhibition; lower doses may also be useful for specific applications.

In one embodiment, the at least one Vitamin E component can be comprised in an enriched formulation. “Enriched” means that at least one Vitamin E component is comprised in an amount which is higher than in the normal mixture comprising all other Vitamin E components. For example, tocotrienol isolated from, e.g., palm oil, comprises γ-tocotrienol and σ-tocotrienol in an amount of less than 10 wt. % based on the total weight of the oil. Thus, with respect to the embodiments of the present invention, an “enriched” formulation means any formulation comprising a specific Vitamin E component, for example, γ-tocotrienol or σ-tocotrienol or a mixture of γ-tocotrienol and σ-tocotrienol, in an amount of more than 0.1% of the respective Vitamin E component based on the total weight of the formulation (or composition).

In another embodiment, the enriched formulation can comprise a specific Vitamin E component in an amount of about 0.1 wt. %, 0.5 wt. %, 1 wt. %, 2 wt. %, 3 wt. %, 4 wt. %, 5 wt. %, 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 92 wt. %, 94 wt. %, 96 wt. %, 97 wt. % or 98 wt. % total Vitamin E component content based on the total weight of the enriched formulation.

In one embodiment, the composition referred to herein can further include an UV-blocking agent. In the context of this embodiment, an UV-blocking agent refers to any compound or substance which itself acts to blocks out ultraviolet radiation, such as zinc oxide, titanium oxide, oxybenzone, azobenzone and avobenzone.

The amount of composition referred to herein can be administered to the patient at any appropriate concentration as long as the composition provides the intended desired effect and does not cause an adverse effect to the patient. In some embodiments, the amount of composition administered to the patient can be between about 1 mg and about 1500 mg; between about 1 mg and about 1200 mg; about 1 mg and about 1000 mg; about 1 mg and about 800 mg; about 1 mg and about 500 mg; about 10 mg and about 1500 mg; about 25 mg and about 1500 mg; about 30 mg and about 1500 mg; about 30 mg and about 1000 mg; about 40 mg and about 1000 mg; about 10 mg and about 800 mg; or between about 10 mg and about 500 mg. In another embodiment, the composition can be administered in an amount to obtain a serum level concentration in the blood of a patient between about 0.5 μM to about 50 μM; between about 0.5 μM to about 50 μM; between about 1 μM to 30 μM or between about 10 μM to 30 μM. Within the context of this embodiment, the amount of Vitamin E component used in the composition referred to herein can be between about 0.5 μM to about 80 μM; about 1 μM to about 60 μM; about 2.5 μM to about 50 μM; about 4 μM to about 80 μM; about 5 μM to about 50 μM; about 5 μM; about 10 μM; about 15 μM; about 20 μM; about 30 μM; or about 50 μM. The amount of the additional component different from the Vitamin E component that has anti-tyrosinase activity and/or anti-melanogenesis activity used in the composition referred to herein can be between about 1 μM to about 5000 μM; about 1 μM to about 4500 μM; about 1 μM to about 3500 μM; about 1 μM to about 3000 μM; about 1 μM to about 2000 μM; about 1 μM to about 1000 μM; about 1 μM to about 500 μM; about 1 μM to about 250 μM; about 5 μM to about 5000 μM; about 10 μM to about 5000 μM; about 20 μM to about 5000 μM; about 50 μM to about 5000 μM; about 100 μM to about 5000 μM; about 200 μM to about 5000 μM; about 300 μM to about 5000 μM; about 10 μM; about 20 μM; about 50 μM; about 60 μM; about 1000 μM; about 3500 μM; or about 4500 μM. In one embodiment, the patient is an animal. The animal can for example be a mammal such as, but are not limited to a human, pig, horse, mouse, rat, cow, dog or cat.

In one embodiment, the composition can be administered as a softgel, an eyestick, a hard capsule, tablet, gel, dragée, sustained-release formulation, lotion, ointment, gel, spray, thin liquid, body splash, mask, serum, solid cosmetic stick, lip balm, shampoo, liquid soap, bath oil, cologne, hair conditioner, cream, such as moisturizer cream; facial wash, injectable formulation, nanoparticle form or emulsion of nanoparticle or in encapsulated form. In a further embodiment, the composition referred to herein can be used in commercially available dermatological compositions and are not limited to skin whitening creams, moisturizers to mention only a few.

In one embodiment, the composition can be administered in a water soluble form. Thus, when desired, the compositions referred to herein can be water solubilized by the addition of specific compounds. A water solubilized form of a composition referred to herein can be obtained, for example, by formulating it into a solid dispersion. Other methods of formulating water-dispersible or water-soluble tocotrienol forms are disclosed for example in U.S. Pat. No. 5,869,704.

The term “solid dispersion” defines a system in a solid state (as opposed to a liquid or gaseous state) comprising at least two components, wherein one component is dispersed throughout the other component or components. For example, the components of the composition can be dispersed in a matrix comprised of a pharmaceutically acceptable water-soluble polymer(s) and a pharmaceutically acceptable surfactant(s).

The term “solid dispersion” encompasses systems having small particles of one phase dispersed in another phase. These particles are typically of less than 400 μm in size, for example less than 100 μm, 10 μm, or 1 μm in size. When said dispersion of the components is such that the system is chemically and physically uniform or homogenous throughout or consists of one phase (as defined in thermodynamics), such a solid dispersion will be called a “solid solution” or a “glassy solution.” A glassy solution is a homogeneous, glassy system in which a solute is dissolved in a glassy solvent.

Such solid dispersions can be administered via different routes. For example, orally administered solid dosage forms include but are not limited to capsules, dragées, granules, pills, powders, and tablets. Excipients commonly used to formulate such dosage forms include encapsulating materials or formulation additives such as absorption accelerators, antioxidants, binders, buffers, coating agents, colouring agents, diluents, disintegrating agents, emulsifiers, extenders, fillers, flavouring agents, humectants, lubricants, preservatives, propellants, releasing agents, sterilizing agents, sweeteners, solubilizers, and mixtures thereof.

Excipients for orally administered compounds in solid dosage forms can include, but are not limited to agar, alginic acid, aluminium hydroxide, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatine, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, talc, tragacanth, water, safflower oil, sesame oil, sesamin, sesamol, sodium carboxymethyl cellulose, sodium lauryl sulfate, sodium phosphate salts, soybean oil, sucrose, tetrahydro fur fury 1 alcohol, and mixtures thereof.

In one embodiment, a dosage form can comprise a solid solution or solid dispersion of at least one Vitamin E component and at least one additional component different from the Vitamin E component that has anti-tyrosinase activity and/or anti-melanogenesis activity in a matrix. The matrix can comprise at least one pharmaceutically acceptable water-soluble polymer and at least one pharmaceutically acceptable surfactant. Suitable pharmaceutically acceptable water-soluble polymers include, but are not limited to, water-soluble polymers having a glass transition temperature (T_{g}) of at least 50° C., or at least 60° C., or from about 80° C. to about 180° C.

Water-soluble polymers having a T_{g} as defined above allow for the preparation of solid solutions or solid dispersions that are mechanically stable and, within ordinary temperature ranges, sufficiently temperature stable so that the solid solutions or solid dispersions can be used as dosage forms without further processing or be compacted to tablets with only a small amount of tableting aids.

The water-soluble polymer comprised in a dosage form referred to herein is a polymer that can have an apparent viscosity, when dissolved at 20° C. in an aqueous solution at 2% (w/v), of 1 to 5000 mPa s, or of 1 to 700 mPa s, or of 5 mPa s to 100 mPa s.

Water-soluble polymers suitable for use in a dosage form referred to herein can include, but are not limited to homopolymers and copolymers of N-vinyl lactams, especially homopolymers and copolymers of N-vinyl pyrrolidone, e.g. polyvinylpyrrolidone (PVP), copolymers of N-vinyl pyrrolidone and vinyl acetate or vinyl propionate; cellulose esters and cellulose ethers, in particular methylcellulose and ethylcellulose, hydroxyalkylcelluloses, in particular hydroxypropylcellulose, hydroxyalkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide; polyacrylates and polymethacrylates such as methacrylic acid/ethyl acrylate copolymers, methacrylic acid/methyl methacrylate copolymers, butyl methacrylate/2-dimethylamino ethyl methacrylate copolymers, poly(hydroxyalkyl acrylates), poly(hydroxyalkyl methacrylates); polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol; oligo- and polysaccharides such as carrageenans, galactomannans and xanthan gum, or mixtures of one or more thereof.

The term “pharmaceutically acceptable surfactant” as used herein refers to a pharmaceutically acceptable non-ionic surfactant. A dosage form referred to herein comprises at least one surfactant having a hydrophilic lipophilic balance (HLB) value of from 12 to 18, or from 13 to 17, or from 14 to 16. The HLB system attributes numeric values to surfactants, with lipophilic substances receiving lower HLB values and hydrophilic substances receiving higher HLB values.

In one embodiment, a dosage form referred to herein comprises one or more pharmaceutically acceptable surfactants selected from polyoxy ethylene castor oil derivates, e.g. polyoxyethyleneglycerol triricinoleate or polyoxyl 35 castor oil (Cremophor® EL) or polyoxyethyleneglycerol oxystearate such as polyethylenglycol 40 hydrogenated castor oil (Cremophor® RH 40, also known as polyoxyl 40 hydrogenated castor oil or macrogolglycerol hydroxystearate) or polyethylenglycol 60 hydrogenated castor oil (Cremophor® RH 60); or a mono fatty acid ester of polyoxy ethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40), or polyoxyethylene (20) sorbitan monolaurate (Tween® 20). Other surfactants including those with HLB values of greater than 18 or less than 12 may also be used, e.g., block copolymers of ethylene oxide and propylene oxide, also known as polyoxyethylene polyoxypropylene block copolymers or polyoxyethylene polypropyleneglycol, such as Poloxamer® 124, Poloxamer® 188, Poloxamer® 237, Poloxamer® 388, or Poloxamer® 407.

Where two or more surfactants are used, the surfactant(s) having an HLB value of from 12 to 18 preferably accounts for at least 50% by weight, more preferably at least 60% by weight, of the total amount of surfactants used.

A dosage form referred to herein can also include additional excipients or additives such as flow regulators, lubricants, bulking agents (fillers) and disintegrants. Such additional excipients may comprise, without limitation, from 0% to 15% by weight of the total dosage form.

Dosage forms referred to herein can be provided as dosage forms consisting of several layers, for example laminated or multilayer tablets. They can be in open or closed form. “Closed dosage forms” are those in which one layer is completely surrounded by at least one other layer. Multilayer forms have the advantage that two active ingredients which are incompatible with one another can be processed, or that the release characteristics of the active ingredient(s) can be controlled. For example, it is possible to provide an initial dose by including an active ingredient in one of the outer layers, and a maintenance dose by including the active ingredient in the inner layer(s). Multilayer tablets types may be produced by compressing two or more layers of granules.

Furthermore, a film coat on the tablet can contribute to the ease with which a tablet can be swallowed. A film coat also improves taste and provides an elegant appearance. If desired, the film-coat may be an enteric coat. The film-coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than 5% by weight of the dosage form.

Other specific forms of formulating the compositions referred to herein, include, but are not limited to native oil liquids of tocotrienols, such as palm oil, which can be used for the manufacture of a soft gel, a water soluble emulsion liquid form, which can be used for the manufacture of soft drinks, a cold water dispersible powder, which can be used for the manufacture of soft capsules and tablets, or beadlets, which can be used for the manufacture of hard capsules.

For the manufacture of the compositions referred to herein in form of water soluble emulsion liquid, tocotrienol liquids are used as starting material to which one adds glycerine and blends of emulsifiers. Afterwards the mixture is homogenized into an emulsion.

Examples for emulsifiers which can be used for the formulation of water soluble emulsion liquid include, but are not limited to glycerine fatty acid esters, acetic acid esters of monoglycerides, lactic acid esters of monoglycerides, citric acid esters of monoglycerides, succinic acid esters of monoglycerides, diacetyl tartaric acid esters of monoglycerides, polyglycerol esters of fatty acids, polyglycerol polyricinoleate, sorbitan esters of fatty acids, propylene glycol esters of fatty acids, starch derivatives, surfactants, sucrose esters of fatty acids, calcium stearoyl di lactate, lecithin, or enzyme digested lecithin/enzyme treated lecithin.

Cold water dispersible powders of the compositions referred to herein can be manufactured by providing tocotrienol oil liquids as starting material. Emulsifiers, such as modified corn starch, maltodextrin, cyclodextrins or corn starch, are added to the tocotrienol oil. The mixture can afterwards be spray dried into a dry powder.

Beadlets comprising compositions referred to herein can be obtained by providing tocotrienol oil liquids as starting material. Afterwards, gelatine, corn starch, sucrose and ascorbyl palmitate are added in one embodiment to the tocotrienol oil. The mixture is spray dried into dry beadlets.

Injectable formulations which allow the introduction and delivery of the above compositions into the circulatory system of the animal body via subcutaneous, intramuscular or intraperitoneal (i.p.) injections in precisely calculated dosages. Propylene glycol is a commonly used solvent for such formulations. In another embodiment the compositions are formulated in a water-in-oil formulation.

The composition referred to herein can be administered into the patient via any suitable means as long as the intended therapeutic or cosmetic effect is achieved. In some embodiments, the composition can for example be administered into the patient via topical or intra-ocular or systemic or oral, or rectal or transmucosal, or intestinal or intramuscular, or subcutaneous, or intramedullar, or intrathecal, or direct intraventricular, or intravenous, or intravitreal, or intraperitoneal, or intranasally administration.

The pharmaceutical composition further includes a pharmaceutically acceptable carrier or excipient. The “carrier” or “excipient” can include any pharmaceutically acceptable carrier as long as the carrier is compatible with other ingredients of the formulation and not injurious to the patient. Accordingly, pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. The pharmaceutically acceptable carrier or excipient can be any of cellulose, hydroxymethylcellulose, cellulose acetate phthalate (CAP) gellan gum, polyalcohol, polyvinyl alcohol, hyaluronic acid, polyacrylic acid, carbopol polymer, poloxamer, poly(oxyethylene) and poly(oxypropylene) and block copolymers thereof, polyethylene oxide, polycarbophil, chitosan, cyclodextrin, liposome, nanoparticle, microparticle including microsphere and nanosphere, niosome, pharmacosome, collagen shield, ocular film or combinations thereof.

In another aspect of the present invention, there is provided a pharmaceutical composition comprising at least one Vitamin E component and at least one additional component different from the Vitamin E component that has anti-tyrosinase activity and/or anti-melanogenesis activity, wherein the at least one additional component different from the Vitamin E component is selected from the group consisting of an antioxidant, a tyrosinase inhibitor, a polyphenol, a vitamin, an anti-tyrosinase RNA interference agent, an anti-tyrosinase peptide, or a mixture thereof.

In still another aspect of the invention, there is provided an ointment or cream comprising a pharmaceutical composition as described herein.

In yet another aspect of the present invention, there is provided α product comprising or consisting of acylated 3,4-dihydro-2,5,7,8-tetramethyl-2-(4,8,12-trimethyl-11-tridecenyl)-2H-1-benzopyran-6-ol. In one embodiment, 3,4-dihydro-2,5,7,8-tetramethyl-2-(4,8,12-trimethyl-11-tridecenyl)-2H-1-benzopyran-6-ol can be acetylated.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXAMPLES

### Example 1

### Materials and Methods

B16 mouse melanoma cells were purchased from (ATCC, Manassas, Va., USA).

The base medium for the cell line was Dulbecco's modified Eagle's medium (DMEM). To make the complete growth medium, fetal bovine serum (final concentration, 10%) was added to the base medium. Tocotrienol (T3) and tocopherol (TP) isomers and the palm tocotrienol-rich fraction (TRF) were purified from a palm fatty acid distillate (PFAD) using molecular distillation and Novasep® equipment (Novasep, Pompey, France). The extraction facility is located in Tuas, Singapore. The PFAD feed was purchased from Kuala Lumpur Kepong Berhad (Kuala Lumpur, Malaysia). The purity of the vitamin E isomers was verified by HPLC and gas chromatography (GC) percentage peak area.

### Example 2

### Synthesis of Palm Vitamin E Acetate

10 g of palm VE was dissolved in 20-50 ml alcohols or alcohol/water mixture (solvent) and was cooled down to −20° C. to −40° C. to remove impurities such as squalene, and free fatty acids (FFA) After the solvent was removed, the purified palm VE was mixed with 30-100 ml of acetic anhydride and 0.1-0.3 g of trimethyl amine was added. The mixture was stirred under N₂ with a temperature cycle from 50° C. to 100° C. for 1 to 9 hours in the presence of the catalyst pyridine (O'Byrne et al, Studies of LDL oxidation following alpha-, gamma, or delta-tocotrienyl acetate supplementation of hypercholesterolemic humans. Free Radical Biology & Medicine, 29, 834-45, 2000). Excess acetic anhydride and its corresponding acid and the catalyst were removed by vacuum distillation in the range of 60° C. to 100° C. The residue was dissolved in 20 ml hexane and washed by 20 ml dilute NaHCO₃ aqueous solution followed by 20 ml water. The obtained organic layer was dried under reduced pressure to afford dark yellow oil. These residual VE acetates were further purified by short path distillation (SPD) at 160° C. to 240° C. under less than 0.01 m bar as described in U.S. Pat. No. 4,517,057. A clear yellow oil with VE acetates content was obtained and no free. VE was detected. The total yield was around 85%.

### Example 3

### UV Spectrophotometry and Emitter

An Agilent 8453 UV-visible spectrophotometer with a photodiode array (PDA) (Agilent Technologies, Santa Clara, Calif., USA) was used for the study of UV spectra, ranging from 200-500 nm, using a 1 cm cuvette. The UVB source for cell irradiation was provided using a UVP UVM-57 handheld UV lamp (UVP Inc, Upland, Calif., USA).

### Example 4

### Cell Viability Study and Time Course Experiment

For the cell viability study, 5×10³ cells re-suspended in 100 μl of medium were plated into each well of a 96-well plate. The cells were then treated with different concentrations of the vitamin-E isomers for 24 hrs. After the treatment, 20 μl of MTT solution (1 mg/ml in PBS; Sigma-Aldrich, St. Louis, Mo., USA) was added to each well and the cells were incubated at 37° C. for 2 hrs. The formazan crystals were then re-suspended in 200 μl of DMSO and the intensity at 595 nm was measured. Each experiment was repeated three times in triplicate wells and the growth curves showed the means and standard deviations.

### Example 5

### Western Blotting

Detailed protocols have been described previously in Chu et. al., A novel anticancer effect of garlic derivatives: inhibition of cancer cell invasion through restoration of E-cadherin expression. Carcinogenesis, 2006, 27, 2180-9. Briefly, cell lysates were prepared by suspending cell pellets in lysis buffer (50 mmol/l Tris-HCl [pH 8.0], 150 mmol/l NaCl, 1% NP40, 0.5% deoxycholate, 0.1% SDS, 1 mg/ml aprotinin, 1 μg/ml leupeptin, and 1 mmol/l phenylmethylsulfonyl fluoride). The protein concentration was measured using the DC Protein Assay kit (Bio-Rad, Hercules, Calif., USA). An equal amount of protein (30 μg) was loaded onto a 10% SDS polyacrylamide gel for electrophoresis, then transferred onto a polyvinylidene difluoride membrane (Amersham, Piscataway, N.J., USA). The membrane was then incubated with primary antibodies for 1 hr at room temperature against tyrosinase, Id1, and beta-actin (Santa Cruz Biotechnology, Inc., Santa Cruz, Calif., USA). After incubation with appropriate secondary antibodies, signals were visualized by an ECL Western blotting system (Amersham). The expression of β-actin was assessed as an internal loading control for total cell lysate.

### Example 6

### Reverse Transcriptase PCR

Total RNA was isolated using Trizol reagent according to the manufacturer's protocol (Invitrogen, Carlsbad, Calif., USA). cDNA was synthesized using the SuperScript First Strand Synthesis System (Invitrogen, Carlsbad, Calif., USA) and was then amplified by PCR with tyrosinase-specific primers (forward primer, tyrosinase-S,5′-TTCAACCCTTTTCTATGTCC-3′ [−2236/−2217] (SEQ ID NO: 1) and reverse primer, tyrosinase-AS,5′-TCATACAAGATCTGCACCAA-3′ [+63/+42]) (SEQ ID NO: 2) and GAPDH specific primers: 5′ to 3′F ATGACATCAAGAAGGTGGTG (SEQ ID NO: 3); 5′ to 3′R CATACCAGGAAATGAGCTTG (SEQ ID NO: 4). The PCR cycling protocol was as follows: 30 cycles for 10 min at 95° C., 30 sec at 95° C., 30 sec at 55° C., 1 min at 72° C., and 10 min at 72° C. Glyceraldehyde 3-phosphate dehydrogenase (GAPDH) was amplified as an internal control. The PCR products were electrophoresed on a 2% agarose gel and analyzed using a gel documentation system.

### Example 7

### Melanogenic Assays

B16 melanoma cells (5×10⁶ cells/well) were incubated in 6-well plates for at least 16 hrs before being subjected to compound treatments (20 μM γT3 and δT3; palm TRF, sodium lactate, and kojic acid at 0.05%, 0.5%, and 1%, respectively; and 0.05-5 ng/ml retinoic acid; Sigma-Aldrich) for various treatment periods. At different time points, cells were harvested using trysin-EDTA and the cell pellets were washed once with phosphate-buffered saline (PBS). A sample amount from each compound treatment group was divided into two equal parts. These cell pellets were then stored at −80° C. prior to the measurement of the tyrosinase activity and melanin content.

### Example 8

### Tyrosinase Activity

Harvested cell pellets were homogenized in RIPA buffer (10 mM Tris-HCl [pH 7.5], 1% NP-40, 0.1% sodium deoxycholate, 0.1% SDS, 150 mM sodium chloride, and 1 mM EDTA) with protease inhibitors and placed on ice for 30 min. The supernatants were collected after centrifuging the samples at 15,000 g at 4° C. for 30 min. For every 100 μl of supernatant, 200 μl of 0.3% L-DOPA (Sigma-Aldrich) was added. All samples, including a blank control of RIPA buffer and 0.3% L-DOPA, were incubated at 37° C. for 20 min. The dopachrome formation from each sample was then measured at 475 nm. The absorbance percentage values of the treated groups in comparison to untreated controls were calculated against per μg of the total protein content, which was determined using the DC protein assay (Bio-Rad, Hercules, Calif., USA).

### Example 9

### Melanin Content

Harvested cell pellets were dissolved in 1 ml of 1N sodium hydroxide before incubation at 80° C. The melanin content was then immediately measured at 420 nm. The absorbance percentage values of the treated groups in comparison to untreated controls were calculated against per μg of the determined total protein content.

### Example 10

### Establishment of the B16 Xenograft Model

The experimental protocol was approved by the IACUC Committee of the A-STAR Biological Resource Centre (BRC) at Biopolis (IACUC no. 080302). Male BALB/c athymic nude mice (4-5 weeks old, 18-22 g) were purchased from The Jackson Laboratory (Bar Harbor, Me., USA). Mice were housed in Department 1 of the Biological Resource Centre (Biopolis, Singapore) under the standard condition (20.8±2° C., 55±1% relative humidity, 12 hrs light/dark cycle) with rodent diet (Harlan Laboratories, Inc., Indianapolis, Ind., USA) and chlorinated reverse osmosis water supplied in a pathogen-free environment. Briefly, B16 cells were pre-treated with 20 μM of γ- and δ-T3, or palm TRF for 1 week. Then, 5×10⁵ pre-treated B16 cells in 100 μl serum-free DMEM were injected subcutaneously into the flank of nude mice using a 1-ml syringe with a 26-gauge needle (Becton Dickinson, Franklin Lakes, N.J., USA). This was followed by a 2-week oral supplementation of γ- and δ-T3, or palm TRF at the dose of 100 mg/kg/day. Altogether, there are four experimental groups: G1, vehicle control; G2, γT3 (100 mg/kg/d); G3, δT3 (100 mg/kg/d); and G4, full spectrum palm TRF (100 mg/kg/d). The mice were weighed daily and the tumors were measured using a Digital Carbon Fiber Caliper (Fisher Scientific, Pittsburgh, Pa., USA) at the 5^{th} and 14^{th} day after innoculation. After 14 days of treatment, the mice were euthanized by CO₂ inhalation. Blood samples were collected through cardiac bleeding using a 26-gauge needle. Blood samples were incubated at room temperature for 30 min, followed by centrifugation at 4400 rpm for 30 min at 4° C. Serum, as the supernatant, was separated from plasma and stored at −80° C. Tumors, liver, kidney, spleen, lung, heart, and skin were harvested.

### 1.1 Anti-Proliferation Effect of T3 Isomers and Tyrosinase Inhibitors

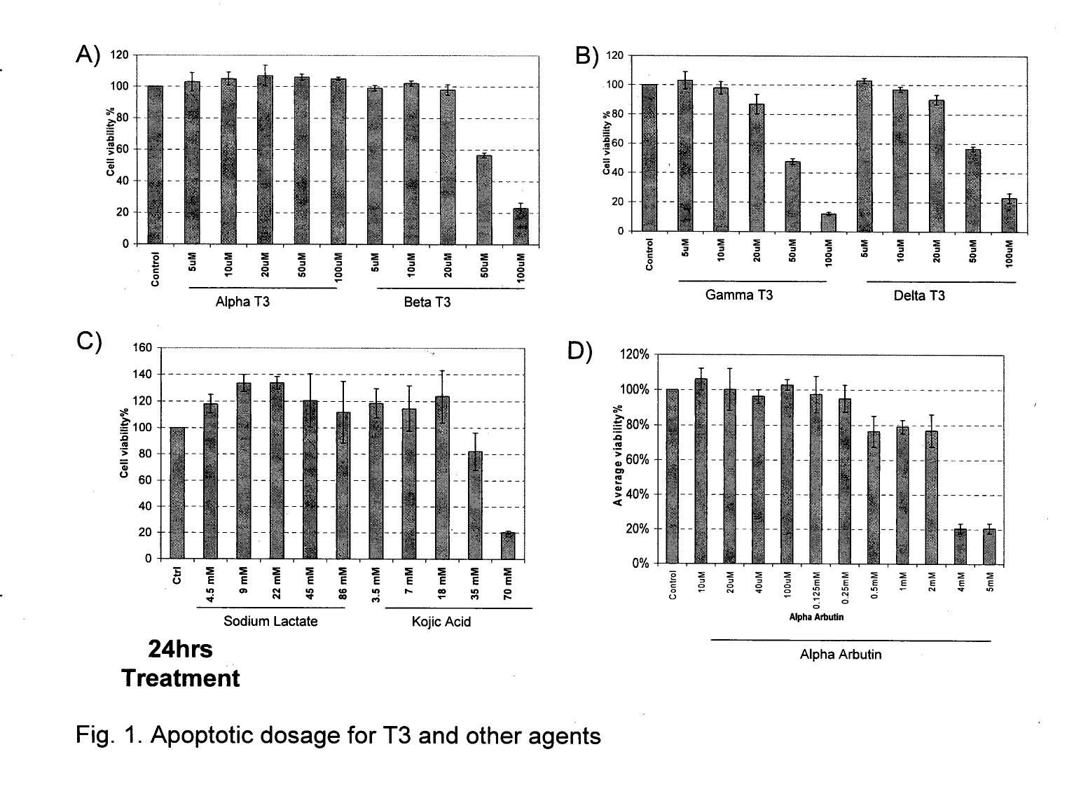

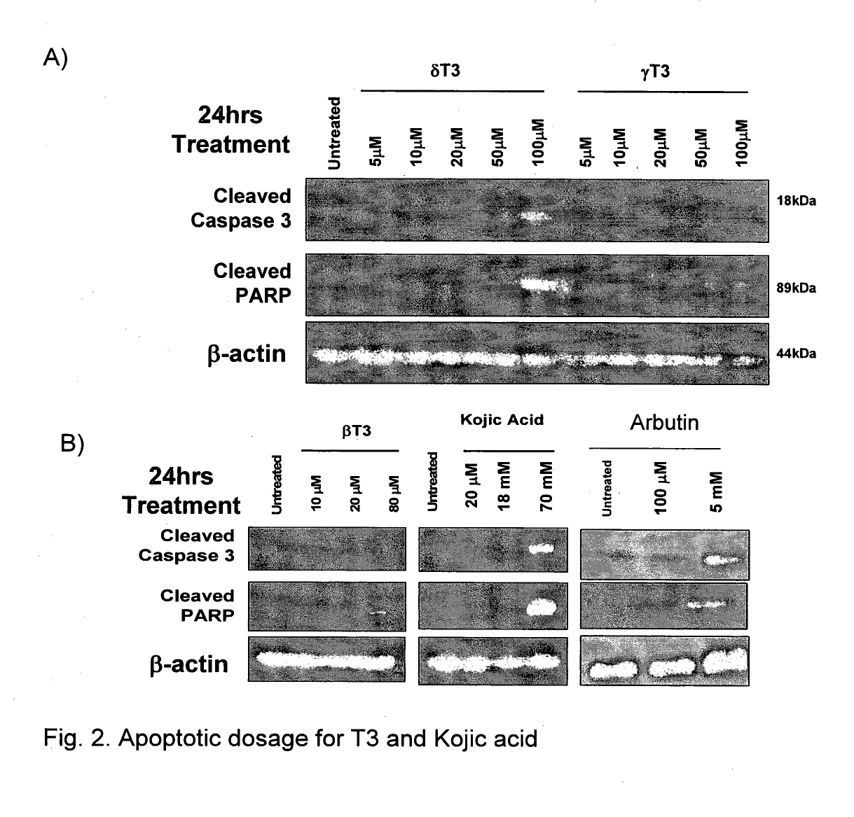

B16 melanoma cells were treated with α-tocotrienol (α-T3), β-tocotrienol (β-T3), γ-tocotrienol (γ-T3), δ-tocotrienol (δ-T3), and sodium lactate, kojic acid and alpha arbutin for 24 hrs at increasing dosages. It has been demonstrated that β-tocotrienol, γ-tocotrienol and δ-tocotrienol inhibited the proliferation rate of B16 melanoma cells in a dose-dependent fashion (FIGS. 1A-B). Among the tyrosinase inhibitors investigated, kojic acid was shown to have an anti-proliferation effect at a concentration ≧0.5% (FIG. 1C). Further investigation using Western blotting revealed that β-, γ-, and δ-T3, and kojic acid induced cellular apoptosis, as evident from the activation of the cleaved caspase 3 and PARP (FIGS. 2A and B).

### 1.2 Anti-Tyrosinase Effect of T3 Isomers and Tyrosinase Inhibitors

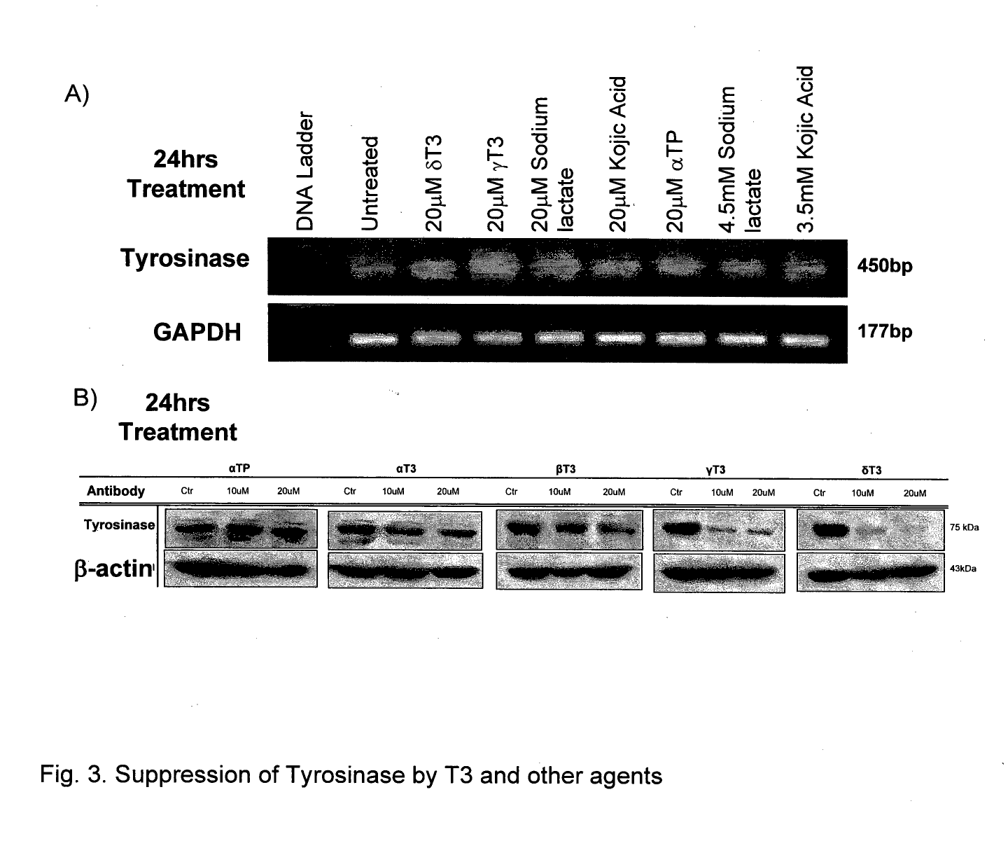

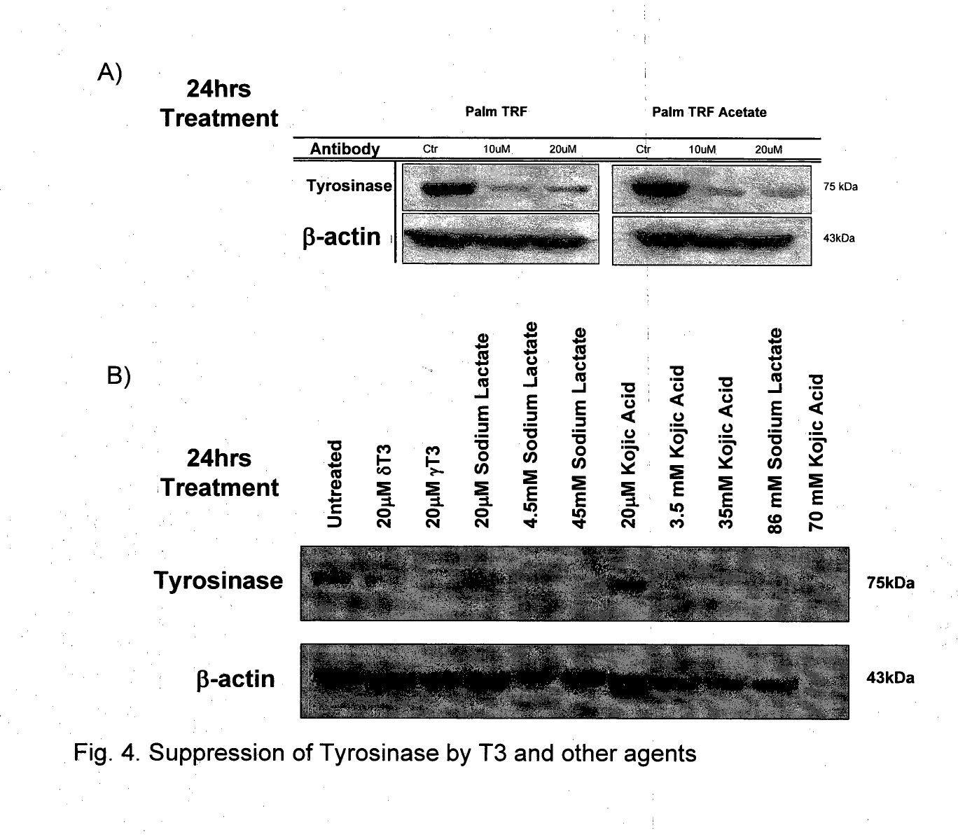

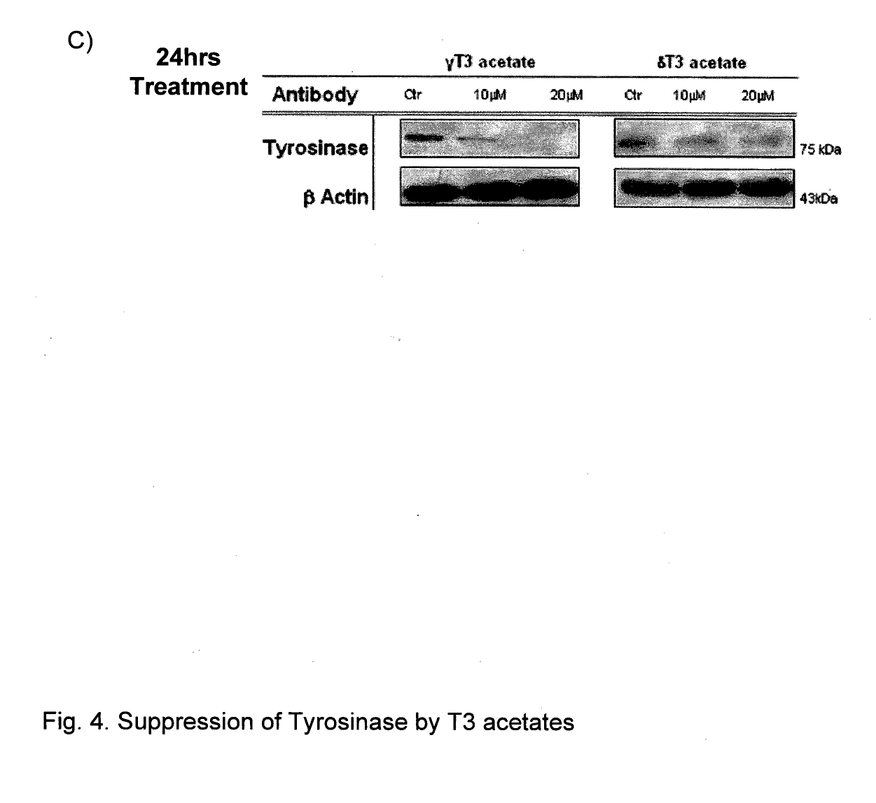

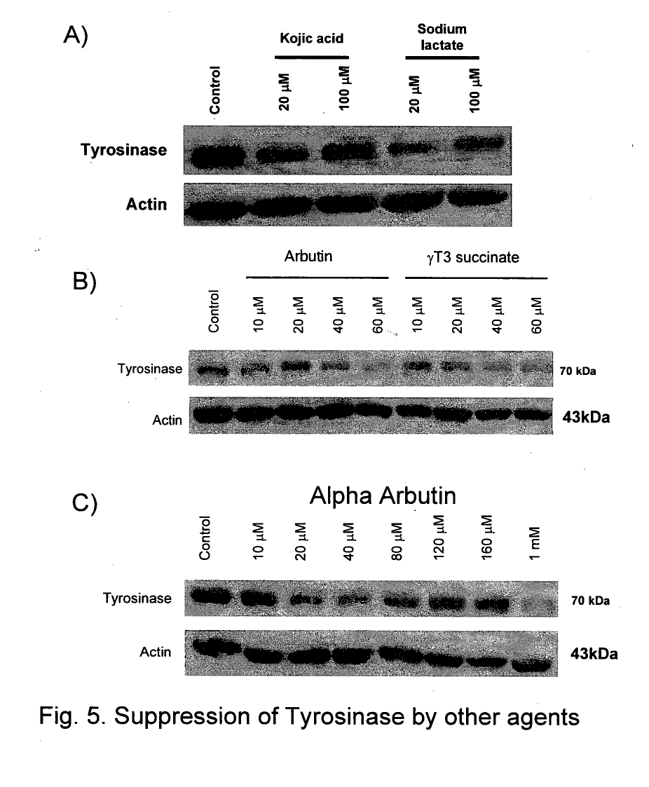

B16 melanoma cells were treated with αTP, γ- and δ-T3 isomers and 2 tyrosinase inhibitors (kojic acid and sodium lactate). The RT-PCR results showed that the mRNA transcript of the tyrosinase gene was not affected by all of the treatments studied (FIG. 3A). However, Western blotting results indicated that 20 μM γ- and δ-T3 treatments resulted in remarkable suppression of tyrosinase protein expression in B16 melanoma cells. The suppression of tyrosinase protein expression was stronger for γT3 (FIGS. 3B and 4B). In contrast, 20 μM treatments with kojic acid and sodium lactate did not result in observable down-regulation of the tyrosinase protein level. Using a higher dose of kojic acid and sodium lactate (≧0.05%), however, led to significant inhibition of tyrosinase protein expression (FIG. 4B).

To study the dose response of tyrosinase suppression by all components of palm TRF, B16 melanoma cells were treated with an increasing dosage of all palm TRF isomers, including αTP. As shown in FIG. 3B, only γ- and δ-T3 induced significant suppression of tyrosinase in a dose-dependent manner. In addition, treatment of B16 melanoma cells with 20 μM of the palm TRF mixture and its acetate also resulted in consistent suppression of tyrosinase protein expression (FIG. 4A). The level of suppression by palm TRF was comparable to that using γ- and δ-T3 isomers alone.

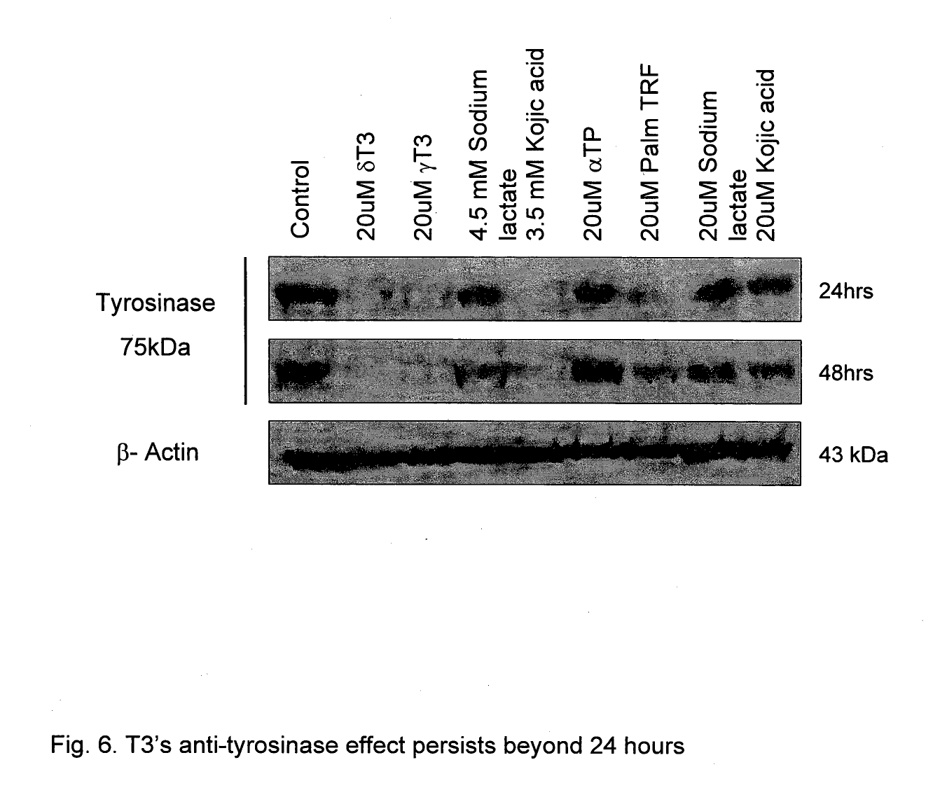

To investigate the time response of tyrosinase suppression by γ- and δ-T3, αTP, and tyrosinase inhibitors, B16 melanoma cells were treated with a single dose of γ- and δ-T3 isomers, αTP, sodium lactate, and kojic acid for up to 48 hrs. Suppression of tyrosinase protein expression by γ- and δ-T3 isomers was shown to be enhanced by increasing the treatment period from 24 to 48 hrs (FIG. 6). However, the opposite observation was determined when the treatment period for sodium lactate and kojic acid increased from 24 to 48 hrs, suggesting that the inhibition by the two agents may be short-lived.

1.3 Tyrosinase Activity and Melanin Content in Cells Treated with T3 Isomers and Tyrosinase Inhibitors

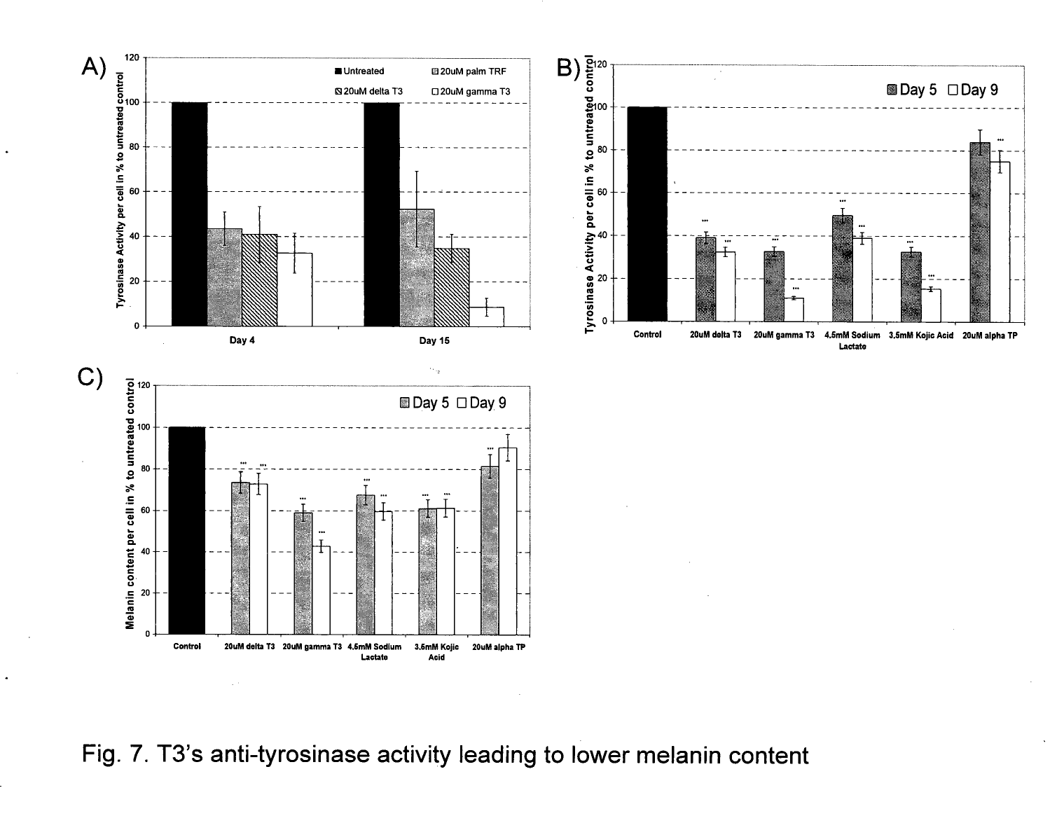

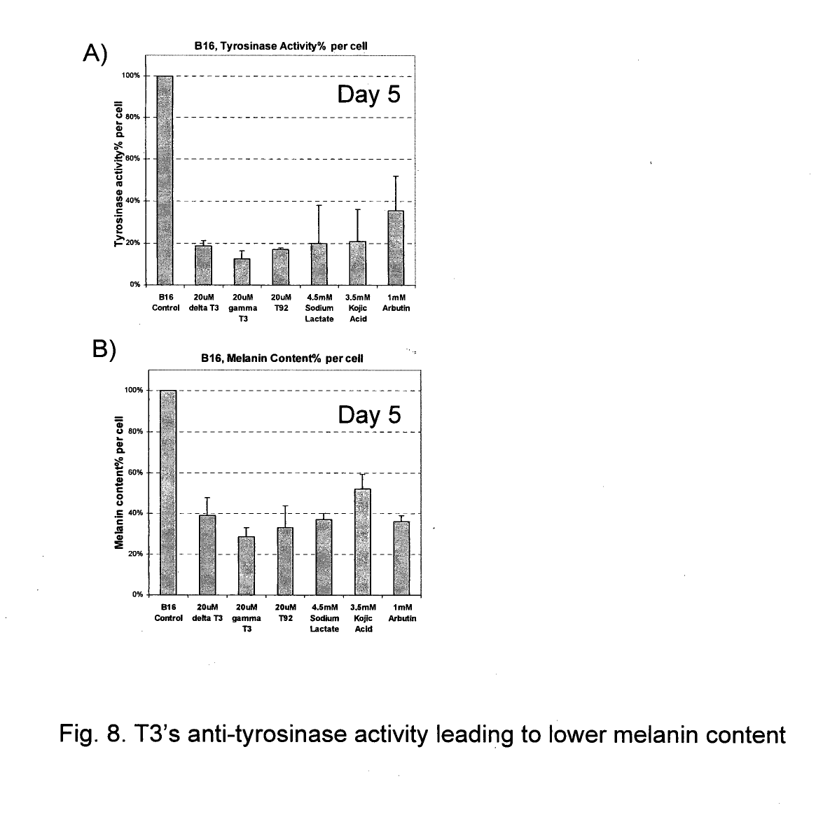

Melanin synthesis rates and total melanin content per cell were determined in control medium (DMEM+10% FBS) and in treated medium. Melanin synthesis rates are represented by the tyrosinase activity assay. The results of representative experiments are given in FIGS. 7 and 8. When tyrosinase activity is normalized for differences in cell growth by dividing total activity by the cell number, B16 melanoma cells treated with γ- and δ-T3 and palm TRF had <45% of the tyrosinase activity in controls. The inhibition of tyrosinase activity remained for up to 15 days after treatment (FIG. 7A). At day 15, B16 melanoma cells treated with γT3 had <15% of the tyrosinase activity compared to controls. FIG. 7B shows that the tyrosinase activity at day 9 following γT3 treatment was comparable to that treated with 0.05% kojic acid. Due to the low γT3 treatment concentration (0.05% kojic acid and sodium lactate are equivalent to 3.52 mM and 4.46 mM, respectively), the inhibition of tyrosinase activity by γ- and δ-T3 was at least 150-fold more potent than kojic acid and sodium lactate. The melanin content of B16 melanoma cell cultures treated with γ- and δ-T3 was 45% and 30% lower than controls at days 5 and 9, respectively (FIG. 7C). The melanin content of B16 melanoma cells following γT3 treatment was marginally lower than the treatment samples using 0.05% sodium lactate and kojic acid (FIG. 7C).

### 1.4 In Vitro and In Vivo Evidence Suggesting Anti-Melanogenesis Properties of γ- and δ-T3

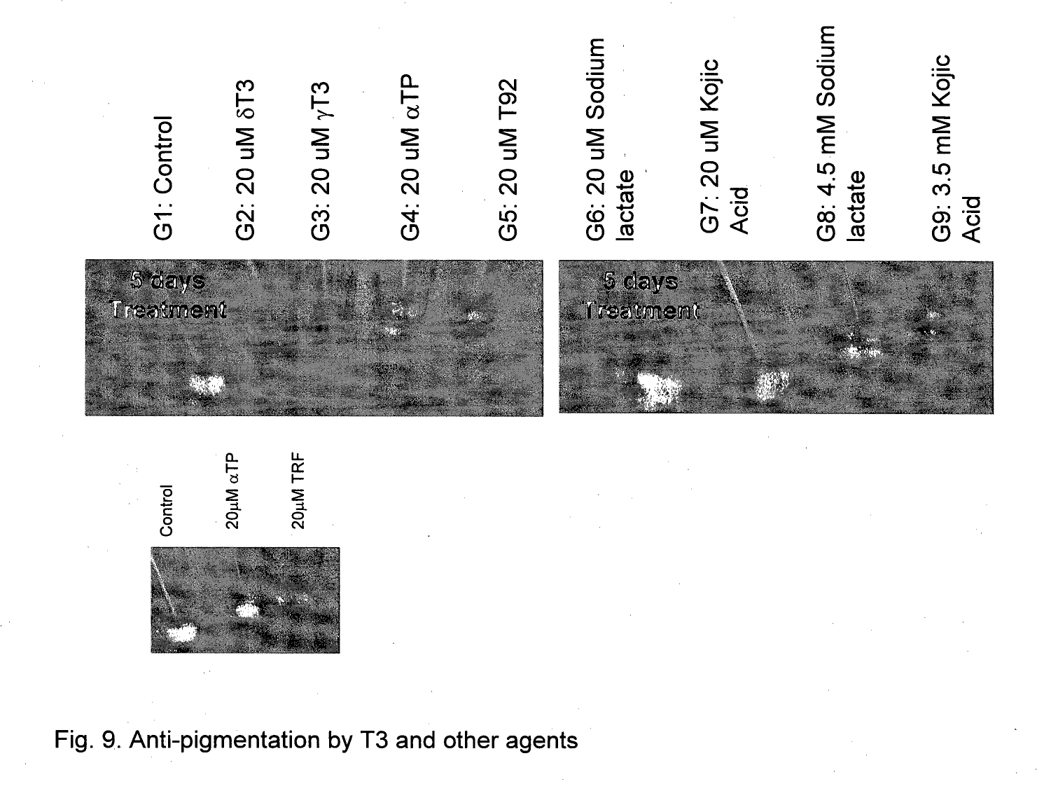

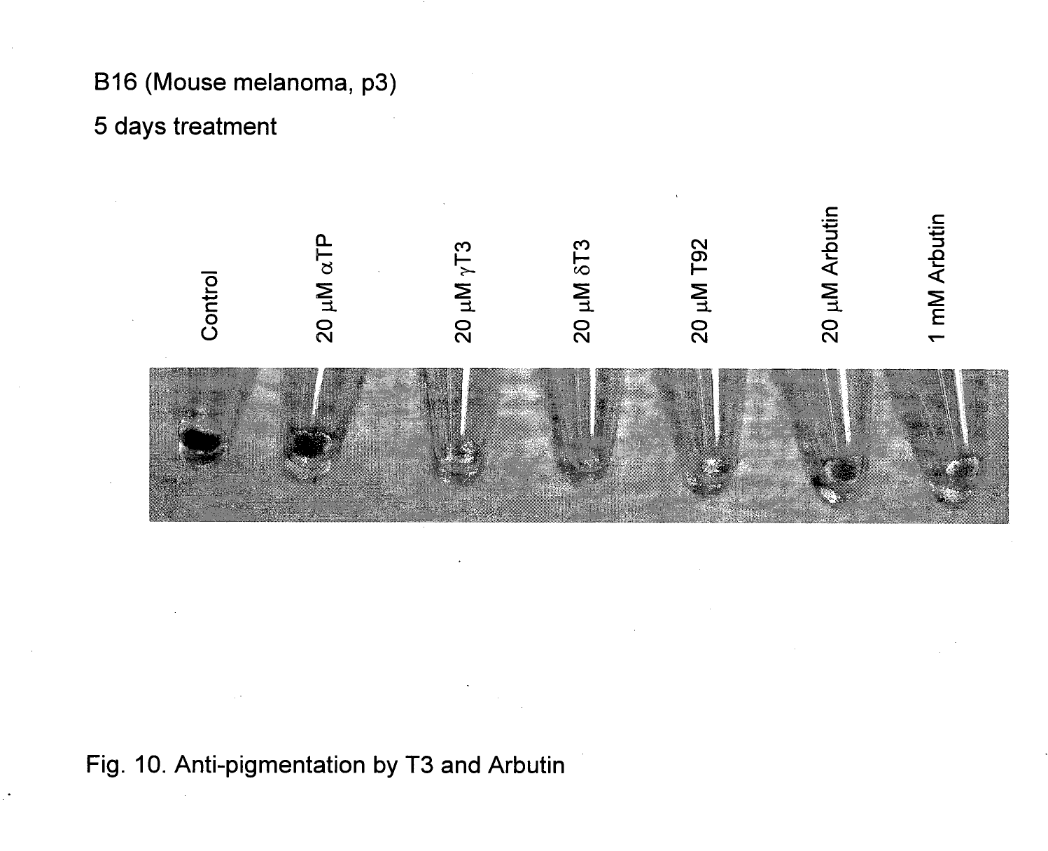

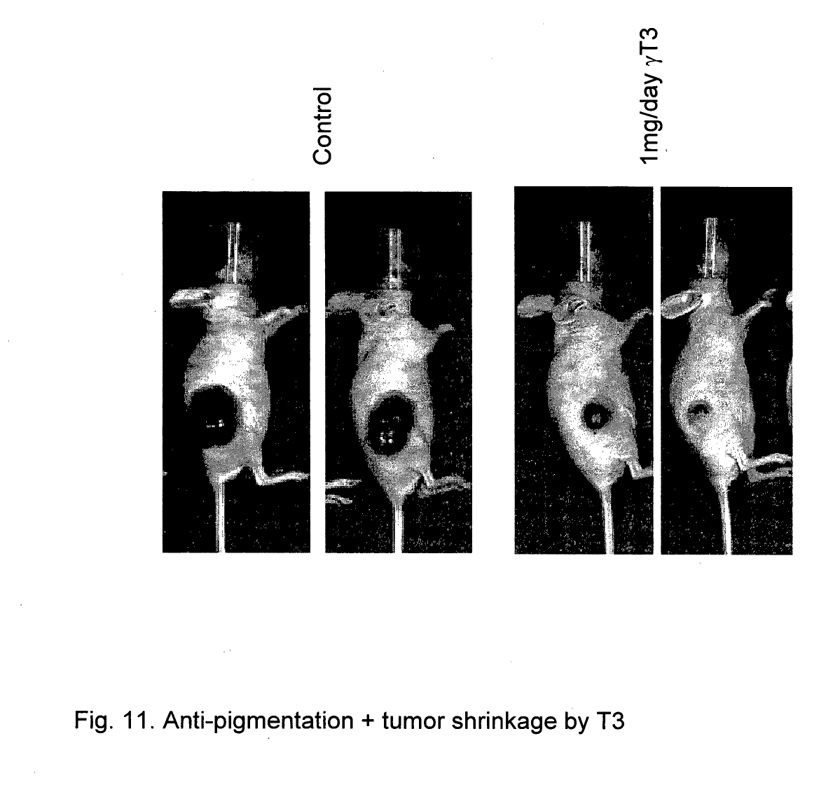

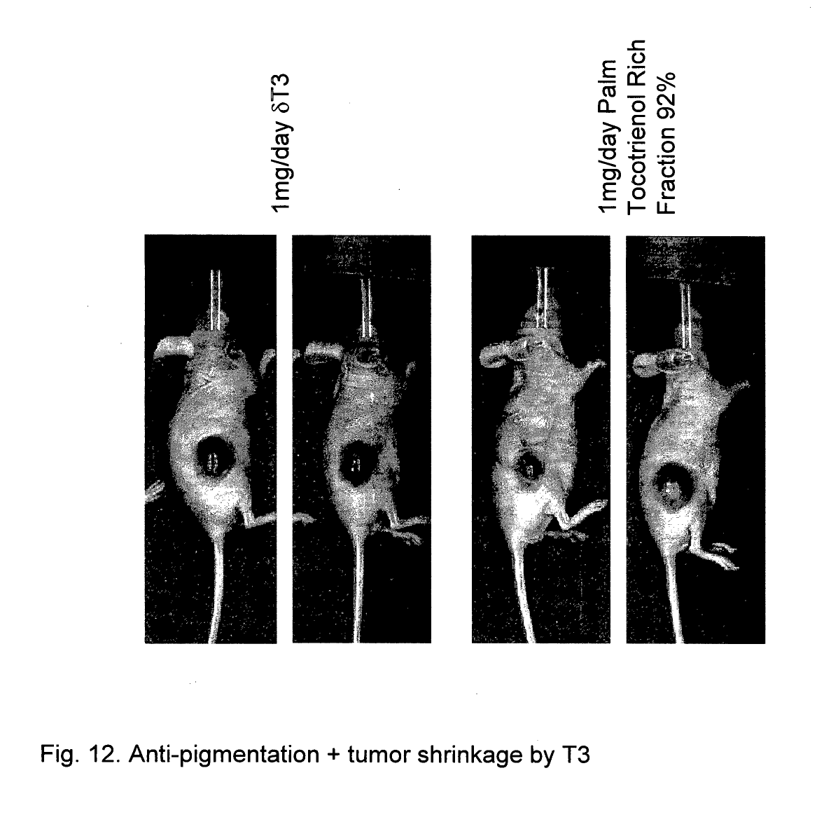

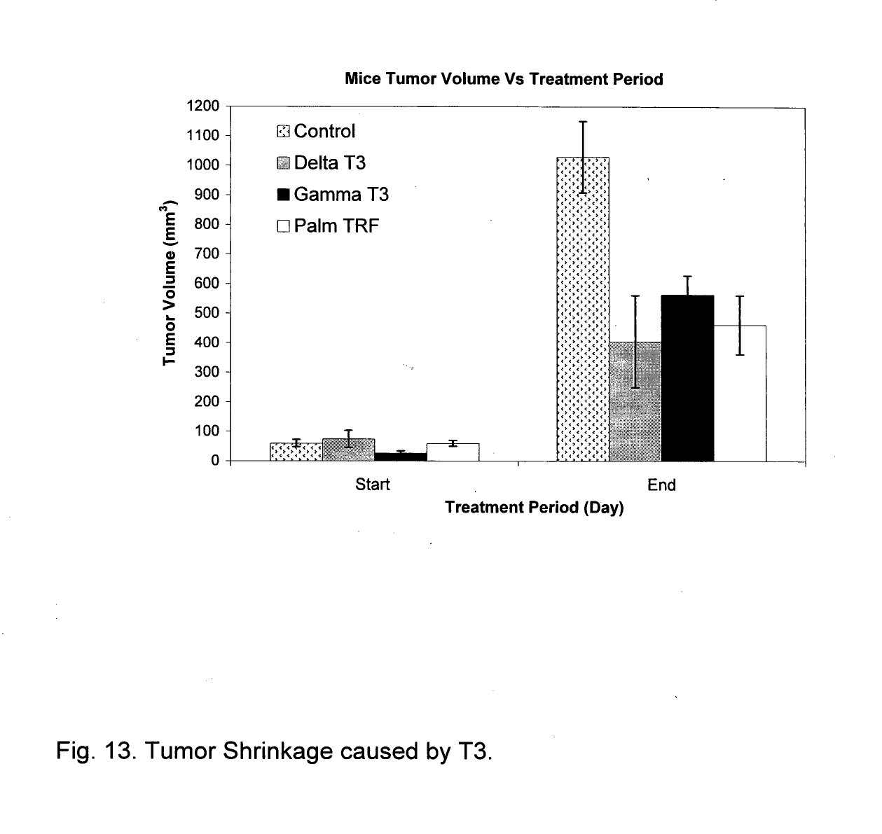

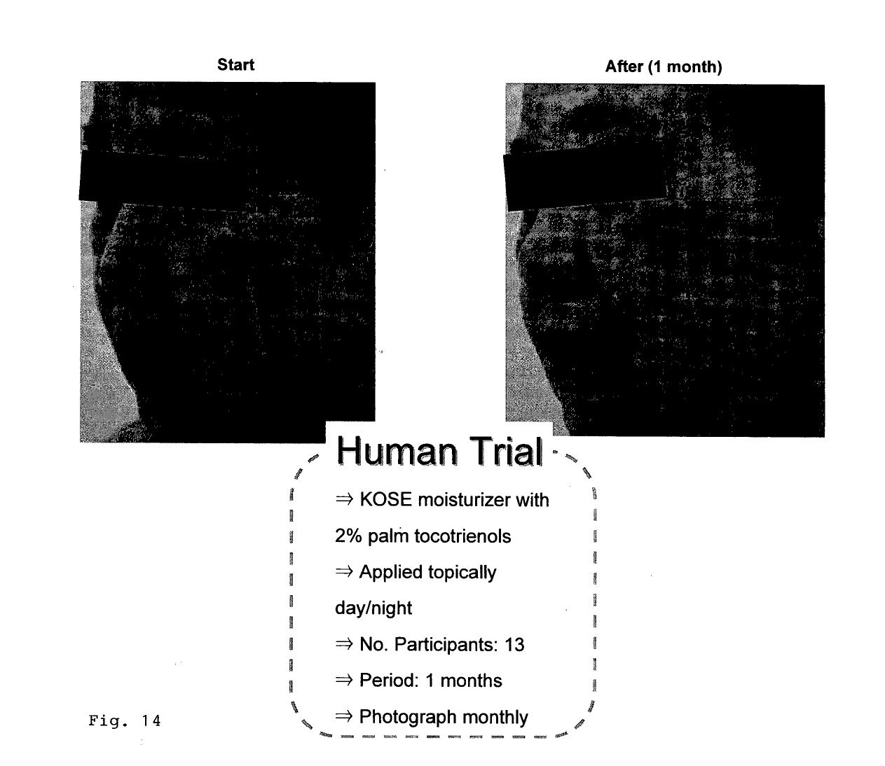

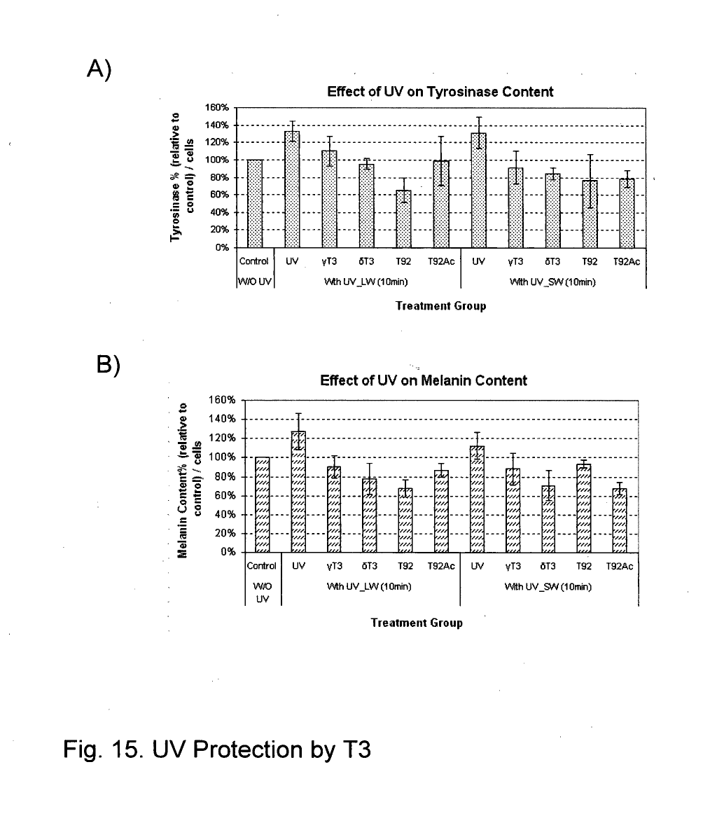

This study reported the ability of γ- and δ-T3, palm TRF, and tyrosine inhibitors to inhibit the induction of melanogenesis in B16 melanoma cells. The pigmentation level of cell pellets and xenografted solid tumors in immunocompromised mice was examined. The results of the experiments are shown in FIG. 9. In FIG. 9, the amount of pigment is demonstrated directly by photographs of cell pellets treated with blank (control), δT3, γT3, αTP, palm TRF, 20 μM sodium lactate, 20 μM kojic acid, 0.05% sodium lactate, and 0.05% kojic acid (left to right panels). Lighter pigmentation was observed in samples treated with γT3, δT3, palm TRF, 0.05% sodium lactate, and 0.05% kojic acid. In FIG. 11, the pigmentation of solid tumors after 14 days of T3 supplementation was not only lighter in colour compared to controls, the tumor size was also significantly smaller for the γT3 and δT3 groups (FIG. 11).

1.5 Synergistic Interaction of γ/δT3 with Kojic Acid and Sodium Lactate

The effect of γ- and δ-T3 alone or in combination with kojic acid, sodium lactate, and retinoic acid was tested. As shown in FIG. 17, the tyrosinase activities per cell following co-treatment with γ- and δ-T3, kojic acid, and sodium lactate are significantly lower than that treated with γ- and δ-T3, kojic acid, or sodium lactate alone. Using Western blotting (FIGS. 17B-C), it was demonstrated that co-treatment of γ- and δ-T3, and kojic acid enhanced the suppression of tyrosinase protein expression when compared to γ- or δ-T3, or kojic acid treatment alone.

### 1.6 γ- and δ-T3 Block Ultraviolet-Induced Melanogenesis

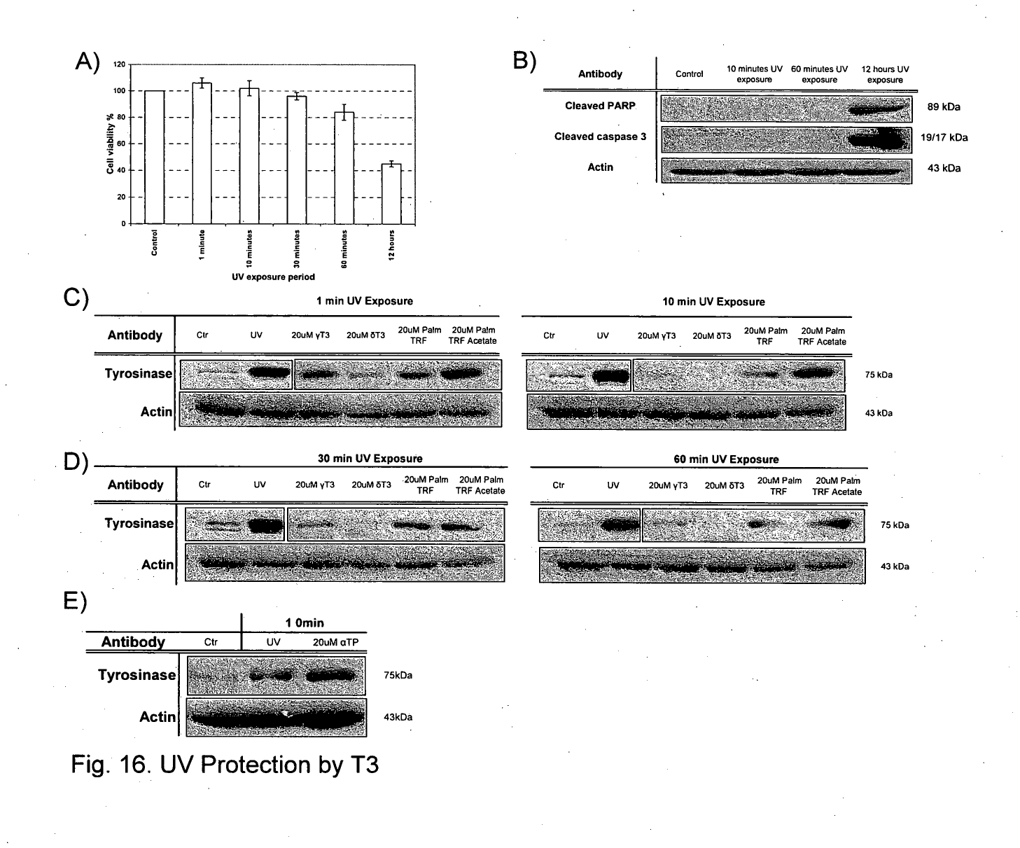

The ability of T3 to also block UVB-induced melanogenesis in B16 melanoma cells was tested. To derive an appropriate UVB irradiation dose, the MTT cell proliferation rate following different doses of UVB exposure was compared. As shown in FIGS. 16A-B, cells exposed to 12 hrs have a reduced cell proliferation rate, as evidenced from the activation of critical apoptotic molecules (cleaved caspase 3 and PARP). Therefore, the UVB exposure of cells was limited to <12 hrs to avoid interference from apoptotic responses. FIGS. 16C-D showed the time-dependent suppression of UVB-induced tyrosinase protein over-expression. Although δT3 was more potent than γT3 in suppressing short-term (<10 min) UVB-induced tyrosinase activation, their long-term inhibitory effect was comparable. Surprisingly, TRF acetate (FIGS. 16C-D) and αTP (FIG. 16E) were not able to block UVB-induced activation of tyrosinase at all time points tested.

1. A method of treating a dermatological condition or preventing a dermatological condition from occurring or for altering the pigmentation of the skin comprising administering to a patient a pharmaceutically effective amount of a composition comprising at least one Vitamin E component and at least one additional component different from the Vitamin E component that has anti-tyrosinase and/or anti-melanogenesis activity, wherein the at least one Vitamin E component is (gamma)γ-tocotrienol, or δ(delta)-tocotrienol, or tocotrienol-rich fraction (TRF), or acylated γ-tocotrienol, or acylated δ-tocotrienol or acylated TRF or mixtures thereof.

2. The method of claim 1, wherein the at least one additional component different from the Vitamin E component is selected from the group consisting of an antioxidant, a tyrosinase inhibitor, a polyphenol, a vitamin, an anti-tyrosinase RNA interference agent, an anti-tyrosinase peptide, or a mixture thereof.

3. The method of claim 1, wherein the at least one additional component different from the Vitamin E component is a skin whitening agent.

4. (canceled)

5. (canceled)

6. (canceled)

7. (canceled)

8. (canceled)

9. (canceled)

10. (canceled)

11. (canceled)

12. (canceled)

13. The method of claim 1, wherein the acylated γ-tocotrienol, or acylated δ-tocotrienol or acylated TRF is an acetylated γ-tocotrienol, or acetylated δ-tocotrienol or acetylated TRF.

14. The method of claim 1, wherein the dermatological condition is a skin disorder.

15. The method of claim 14, wherein the skin disorder is selected from the group consisting of darkening of skin caused by increased melanin, skin hyperpigmentation, skin inflammation, skin acne vulgaris, wound healing, skin photoaging, skin wrinkles, smoker's melanosis, melasma, acanthosis nigricans, Cushing's disease, Addison's disease, linea nigra, mercury poisoning.

16. The method of claim 1, wherein the at least one additional component different from the Vitamin E component is selected from the group consisting of vitamin A, vitamin A derivatives, vitamin B, vitamin B derivatives, vitamin C, vitamin C derivatives, quinones, hydroquinone, lactates, kojic acid, kojic acid derivatives, alpha hydroxy acids, arbutin, glycolic acid, hydroquinone, glutathione, L-glutathione, azelaic acid, glucocorticoids, Mulberry extract, mitracarpus scaber extract, Cucumis sativus extract, licorice extract, pomegranate extract, uva ursi (bearberry) extract, hexamethylene bisacetamide, sodium butyrate, dimethyl sulfoxide, synthetic hydroxyl substituted phenyl naphthalenes and derivatives, monophenols, retinoic acid, tunicamycin, N-acetyl glucosamine, hyaluronic acid, tranexamic, placental extract, ellagic Acid, EDTA, phytic acid, aleosin, thioctic Acid, Protease Activated Receptor (PAR2) and soybean trypsin inhibitor, wherein said monophenols are selected from the group consisting of 2,6-di-tert-butyl-4-methylphenol and 2,6-di-tert-butyl-4-ethylphenol; wherein diphenols is selected from the group consisting of 4,4′-methylenebis(2,6-di-tert-butylphenol), 2,2′-methylenebis(4-ethyl-6-tert-butylphenol).

17. The method of claim 16, wherein the lactate is selected from the group consisting of potassium lactate, sodium lactate, magnesium lactate, ammonium lactate and calcium lactate.

18. The method of claim 17, wherein the at least one additional component different from the Vitamin E component is selected from the group consisting of sodium lactate, hydroquinone, L-glutathione, kojic acid, arbutin and retinoic acid.

19. The method of claim 1, wherein the at least one additional component different from the Vitamin E component is an anti-tyrosinase RNA interference agent selected from the group consisting of miRNA, siRNA and antisense RNA.

20. The method of claim 19, wherein the miRNA is mi434-5P miRNA.

21. The method of claim 1, wherein the composition is administered in an amount of between about 1 mg to about 1000 mg or between about 10 mg and 500 mg.

22. The method of claim 1, wherein the composition is administered in an amount to obtain a serum level concentration in blood in the patient between about 1 to 30 μM or between about 10 to 30 μM.

23. The method of claim 1, wherein the patient is an animal.

24. (canceled)

25. (canceled)

26. The method of claim 1, wherein the composition is administered in a water soluble form; or orally; or topically; or as a softgel, a hard capsule, a tablet, a gel, a dragée, a sustained-release formulation, an ointment, a cream, a moisturizer cream, a facial wash, an eyestick, an injectable formulation, in nanoparticle form or in encapsulated form.

27. (canceled)

28. (canceled)

29. The method of claim 1, wherein the composition further comprises a pharmaceutically acceptable excipient.

30. The method of claim 1, wherein the at least one Vitamin E component is comprised in an enriched formulation, wherein the enriched formulation comprises more than 0.1 wt. % of a specific Vitamin E component based on the total weight of the enriched formulation.

31. (canceled)

32. The method of claim 1, wherein the composition further comprises an UV-blocking agent.

33. The method of claim 32, wherein the UV-blocking agent is selected from the group consisting of zinc oxide, titanium oxide, oxybenzone, azobenzone and avobenzone.

34. A pharmaceutical composition comprising at least one Vitamin E component and at least one additional component different from the Vitamin E component that has anti-tyrosinase activity and/or anti-melanogenesis activity, wherein the at least one additional component different from the Vitamin E component is selected from the group consisting of an antioxidant, a tyrosinase inhibitor, a polyphenol, a vitamin, an anti-tyrosinase RNA interference agent, an anti-tyrosinase peptide, or a mixture thereof.

35. (canceled)

36. (canceled)

37. (canceled)


SEQUENCE LISTING (4 sequences)

SEQ ID NO: 1 - DNA, 20 bp, artificial
  forward primer tyrosinase
ttcaacccttttctatgtcc

SEQ ID NO: 2 - DNA, 20 bp, artificial
  reverse primer tyrosinase
tcatacaagatctgcaccaa

SEQ ID NO: 3 - DNA, 20 bp, artificial
  forward primer GAPDH
atgacatcaagaaggtggtg

SEQ ID NO: 4 - DNA, 20 bp, artificial
  reverse primer GAPDH
cataccaggaaatgagcttg